(19) United States
(12) Patent Application Publication
(10) Pub. No.: US 2023/0287217 A1
(43) Pub. Date: Sep. 14, 2023

(54) Zwitterionic Cell-Permeant and Water-Soluble Rhodamine Dyes for Quantitative Imaging Applications

(71) Applicant: OREGON HEALTH & SCIENCE UNIVERSITY, Portland, OR (US)

(72) Inventors: Summer L. Gibbs, Portland, OR (US); Lei G. Wang, Portland, OR (US); Antonio R. Mantaño, Portland, OR (US)

(21) Appl. No.: 18/248,045

(22) PCT Filed: Oct. 6, 2021
(86) PCT No.: PCT/US2021/053806

§ 371 Date: Apr. 5, 2023

Related U.S. Application Data
(60) Provisional application No. 63/088,427, filed on Oct. 6, 2020.

(51) Int. Cl. C09B 11/24; A61K 49/00; G01N 33/58

(52) U.S. Cl. CPC C09B 11/24; A61K 49/0041; A61K 49/0052; G01N 33/582

(57) ABSTRACT

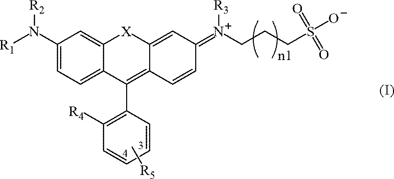

The present invention provides novel Zwitterionic cell-permeant and water-soluble rhodamine dye compounds, pharmaceutical composition comprising them, and methods for their use in quantitative imaging applications, particularly substituted 5,5-dimethyl-10-phenyl-3,5-dihydrodibenzo[b,e]siline and 9- phenyl-3H-xanthene compounds of Formula (I).

### FIELD OF THE INVENTION

The present invention provides novel Zwitterionic cell-permeant and water-soluble rhodamine dye compounds and methods for their use in quantitative imaging applications.

### BACKGROUND OF THE INVENTION

Spatial and temporal measurements in cells and tissues can provide critical information to answer important biological questions, where quantitative imaging of both intracellular and extracellular biomolecular targets precisely and accurately is of pivotal importance in deciphering the complexity of biological systems. Fluorescence imaging has long been used to visualize biological specimens, with significant advances in the development of imaging methodologies and labeling strategies for super resolution microscopy in recent years,¹⁻⁵ providing images with increasing clarity about physiological relevance on a single cell level. Synthetic organic small molecule fluorophores have become the routinely used labels for optical imaging, with applications span from single molecule localization microscopy on a single cell to in vivo whole-body imaging.⁶⁻¹¹ However, classic small molecule fluorophores often times suffer from either poor water solubility or cell-impermeant issues, providing roadblocks for in vitro and in vivo optical imaging applications. Here, we report the design and synthesis of zwitterionic water-soluble and cell-permeable rhodamine and silicon-rhodamine fluorophores, termed Sulfo-Rh and Sulfo-SiRh, which both showed substantially improved stability towards solvent polarity changes comparing to the base fluorophores tetramethyl rhodamine (TMR) and Silicon-substituted TMR (SiTMR). Photophysical property screening also showed sulfonated fluorophore pair exhibited synchronized Stokes shift and brightness, which prompt us to develop robust synthetic sequences for both the N-hydroxysuccinimide (NHS) ester and azido reactive versions, providing utility for subsequent bioconjugation reactions.

Fluorescent proteins (FPs) have become common optogenetic tools for investigating cell structures, functions, and underlying mechanisms of subcellular events. However, FPs are limited to the visible range in the optical spectrum, with high demand for expending the color palette for spectral multiplexing and deep-tissue in vivo imaging.¹²⁻¹⁴ Moreover, the smallest FP is about 17 kDa,¹⁵ not compatible to be used as a fluorescence reporter for small molecule labelling to image either exogenous substrates (e.g., drugs, glycans, etc.) or endogenous substances (e.g., hormone, neurotransmitter, etc.). Among all small molecule fluorophores, rhodamine stands out as a popular class of fluorescent labels due to their higher photostability and brightness as well as easier synthesis compared to the FPs. However, like many other xanthene-base fluorophores, classic rhodamines generally have poor water solubility and tend to aggregate in an aqueous environment. Over the last two decades, there has been a significant effort to address this issue. One such successful chemical engineering approach involves addition of sulfonate groups, affording an array of popular commercially available rhodamine fluorophores, such as ATTO488, Alexa Fluor 532, 546, 568, 549 and 633.¹⁶⁻¹⁸ The modification with sulfonate groups has resulted in water-soluble but cell-impermeant fluorophores due to the negative net charge, which limits their application to the imaging of extracellular targets in the non-permeabilized cells. Additionally, classic rhodamine fluorophores (e.g., tetramethyl rhodamine [TMR], silicon substituted TMR [SiTMR]) exhibit fluorescence on-off switching equilibrium as a result of intramolecular cyclization reaction between the central bridge carbon of the chromophore and the carboxylate group attached at the ortho position of pending phenyl ring. In the context of quantitative fluorescence imaging applications, for instance, drug uptake and distribution monitoring in cells and tissue, the fluorescence on-off switching equilibrium is undesired. Critical information will be missing when the target of interest is bound to the fluorophore at the fluorescence off state (i.e., fluorophores with closed spiro-ring). Our lab has developed novel water-soluble and cell-permeable fluorophores that are insensitive to the environment providing solutions to the limitations stated above.

There remains a need for water-soluble rhodamine-based fluorophores that provide utility in live cell imaging applications and exhibit favorable photophysical properties that support quantitative fluorescence imaging applications.

### SUMMARY OF THE INVENTION

To resolve the classic rhodamine fluorophores’ poor water solubility and cell-impermeant limitations, we have adapted a creative approach to engineer the classic visible to near-infrared rhodamine fluorophores. In particular, the typical nucleophilic group adjacent to the central bridge carbon is replaced with a methyl group, preventing intramolecular cyclization reaction and serving as a protecting group for the central bridge carbon via steric hindrance; the resulting cationic charge is balanced by attaching one sulfonate group to the far end of the N-alkyl group; affording bright zwitterionic water-soluble and cell-permeable rhodamine fluorophores that are insensitive to the polarity of the environment.

In one embodiment, provided herein is a compound of Formula (I):

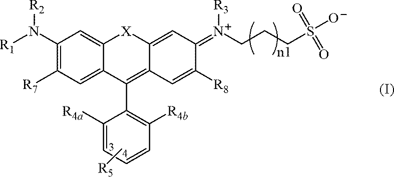

wherein:
X is selected from the group of O, —Si(CH₃)₂—, S, —N(CH₃)—, —C(CH₃)₂—, —P(OOH)—, —P(OPh)—, and —SO₂;
R₁ and R₂ are independently selected from the group of H and C₁-C₃ alkyl, with the proviso that at least one of R₁ and R₂ is not H;
or R₁ and R₂, together with the nitrogen atom to which they are bound, may form an azetidinyl ring, a pyrrolidinyl ring, or a piperidinyl ring, wherein the azetidinyl ring, pyrrolidinyl ring, or piperidinyl ring is substituted by 0, 1, 2, or 3 C₁-C₃ alkyl substituents;
R₃ is selected from the group of H and C₁-C₃ alkyl;
R₄ₐ and R_{4b} are each independently selected from the group of H and C₁-C₃ alkyl;
R₅ is bound to either the 3-position carbon atom or the 4-position carbon atom in the phenyl ring and is selected from the group of H, —C(O)OH, —C(O)O—C₁-C₃ alkyl,

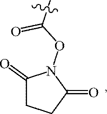

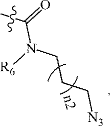

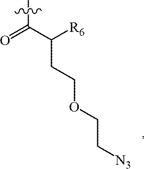

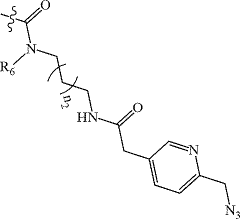

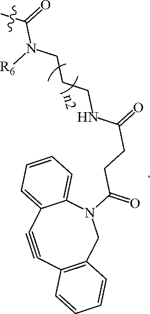

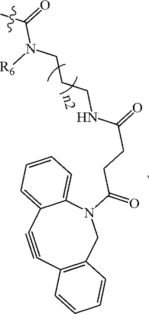

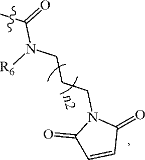

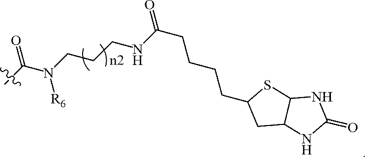

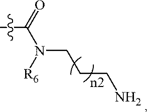

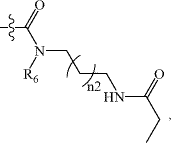

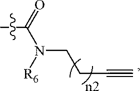

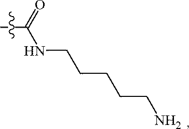

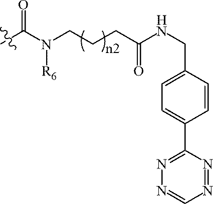

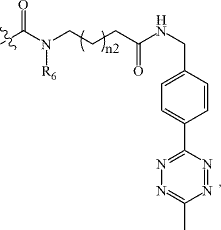

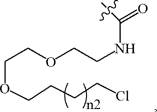

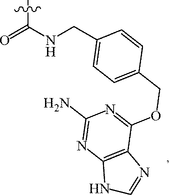

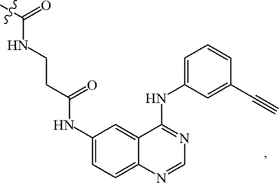

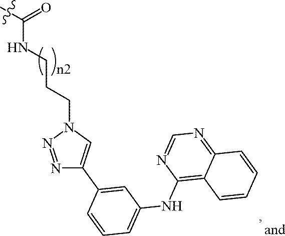

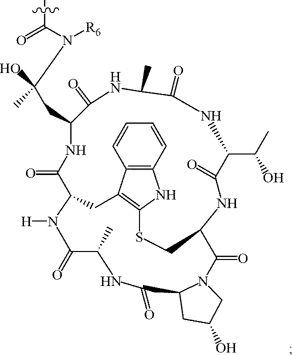

R₆ is selected from the group of H and C₁-C₃ alkyl;
n1 is an integer selected from the group of 0, 1, 2, and 3; and
n2 is an integer selected from the group of 0, 1, 2, 3, 4, 5, and 6;
R₇ is hydrogen;
R₈ is hydrogen;
or R₁ and R₇, together with the nitrogen atom to which R₁ is bound, form a 5-membered or 6-membered fused fully saturated or partially unsaturated nitrogen-containing heterocyclic ring, wherein the 6-membered fused fully saturated or partially unsaturated nitrogen-containing heterocyclic ring may have a further ring oxygen heteroatom to form a fused morpholinyl ring, and further wherein the 5-membered or 6-membered fused fully saturated or partially unsaturated nitrogen-containing heterocyclic ring is substituted by 0, 1, 2, 3, 4, or 5 C₁-C₃ alkyl substituents; or
or R₃ and R₈, together with the nitrogen atom to which R₁ is bound, form a 5-membered or 6-membered fused fully saturated or partially unsaturated nitrogen-containing heterocyclic ring, wherein the 6-membered fused fully saturated or partially unsaturated nitrogen-containing heterocyclic ring may have a further ring oxygen heteroatom to form a fused morpholinyl ring, and further wherein the 5-membered or 6-membered fused fully saturated or partially unsaturated nitrogen-containing heterocyclic ring is substituted by 0, 1, 2, 3, 4, or 5 C₁-C₃ alkyl substituents.

It will be understood that, in the description above, a compound of Formula (A) can include instances in both pairs of R₁/R₇ and R₃/R₈ form fused nitrogen-containing rings.

The wavy line

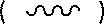

in chemical structures indicates a bond through which the structure shown is bound to another chemical moiety or group.

### BRIEF DESCRIPTION OF THE MANY VIEWS OF THE DRAWINGS

FIG. 1A represents the non-fluorescent lactone form and fluorescent zwitterionic form of silicon-substituted rhodamine (SiTMR).

FIG. 1B represents synthesis of sulfonated versions of TMR and Si-TMR, termed Sulfo-Rh and Sulfo-SiRh.

FIG. 1C presents structures of analogs Sulfo-Rh-NHS, Sulfo-SiRh-NHS, Sulfo-Rh-Azide, and Sulfo-SiRh-Azide.

FIG. 2A depicts general rhodamine fluorophore structures including the classic TMR, SiTMR, and the sulfonated versions.

FIG. 2B graphs normalized absorbance and fluorescence emission spectra of the four fluorophores in PBS buffer.

FIG. 2C provides tabulated spectral properties of all four fluorophores in PBS buffer.

FIG. 2D graphs normalized absorption (left) and fluorescence spectra (right) of fluorophores with varied percentages of dioxane (20-80%) in DI water resulting the polarity of the screening solvent systems changing in a gradient manner.

### DETAILED DESCRIPTION OF THE INVENTION

In separate embodiments, the fused nitrogen-containing rings defined by the two pairs of R₁/R₇ and R₃/R₈ include those of the following Formulas (A-1) through (A-20), wherein, in each embodiment, R₉, R₁₀, R₁₁, R₁₂, R13, R14, R₁₅, R₁₆, R17, R₁₈, and R₁₉, when present, are independently selected from H and C₁-C₃ alkyl and all other variables are as described for Formula (A), above.

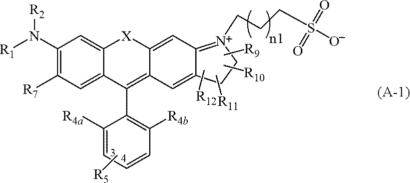

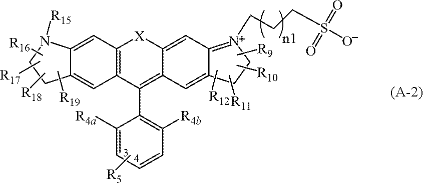

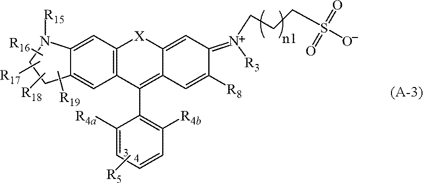

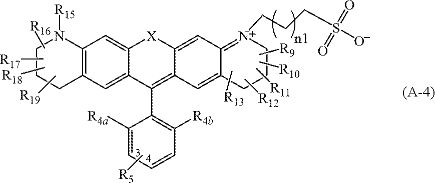

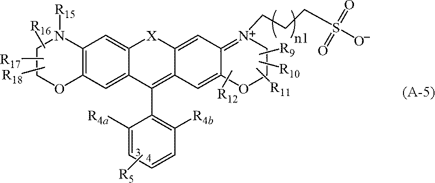

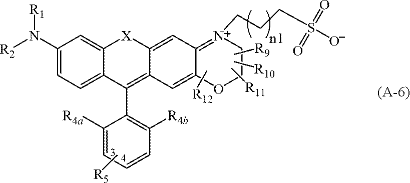

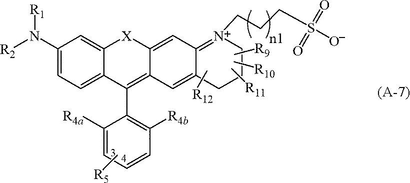

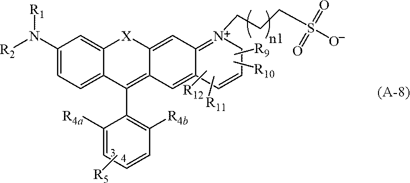

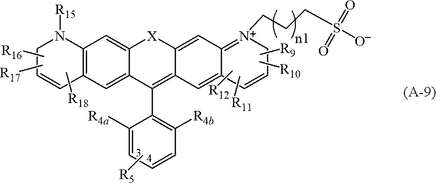

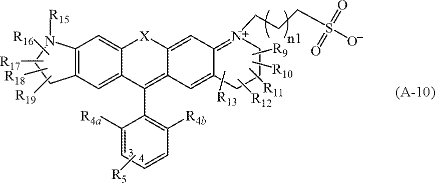

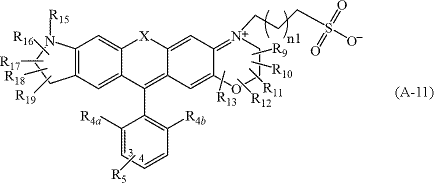

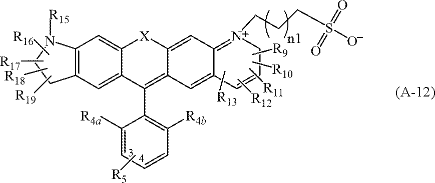

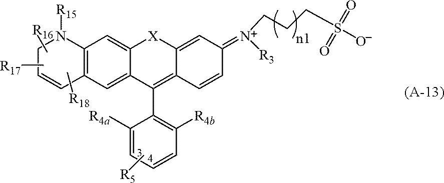

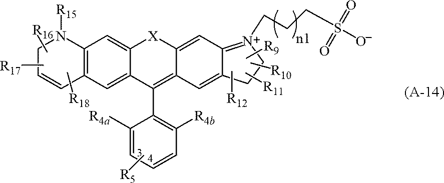

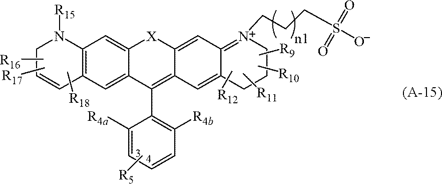

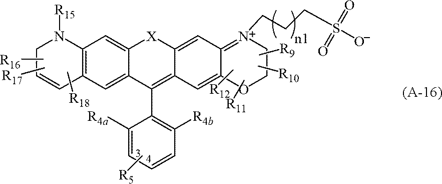

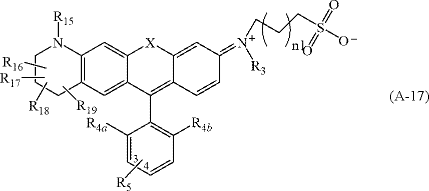

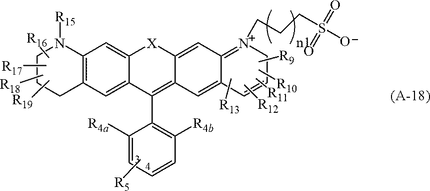

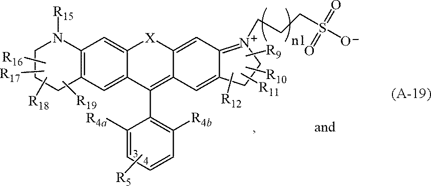

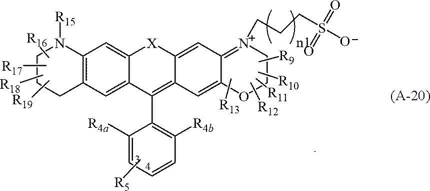

There is an additional embodiment for compounds of each of the Formulas (A-1) through (A-20), wherein R₅ is bound to the 3-position carbon atom of the phenyl ring and all variables and definitions are as indicated from and for Formula (I), above.

There is an additional embodiment for compounds of each of the Formulas (A-1) through (A-20), wherein R₅ is bound to the 4-position carbon atom of the phenyl ring and all variables and definitions are as indicated from and for Formula (I), above.

There is an additional embodiment for compounds of each of the Formulas (A-1) through (A-20), wherein R₅ is bound to the 3-position carbon atom of the phenyl ring; R₄ₐ is hydrogen; R_{4b} is methyl; and all other variables and definitions are as indicated from and for Formula (I), above.

There is an additional embodiment for compounds of each of the Formulas (A-1) through (A-20), wherein R₅ is bound to the 3-position carbon atom of the phenyl ring; R₄ₐ is methyl; R_{4b} is hydrogen; and all other variables and definitions are as indicated from and for Formula (I), above.

There is an additional embodiment for compounds of each of the Formulas (A-1) through (A-20), wherein R₅ is bound to the 4-position carbon atom of the phenyl ring; R₄ₐ is hydrogen; R_{4b} is methyl; and all other variables and definitions are as indicated from and for Formula (I), above.

Also provided for each embodiment listed above, including those from and for Formulas (I) and Formulas (A-1) through (A-20), wherein X is O. Also provided for each embodiment listed above, including those from and for Formulas (I) and Formulas (A-1) through (A-20), wherein X is —Si(CH₃)₂—. Also provided for each embodiment listed above, including those from and for Formulas (I) and Formulas (A-1) through (A-20), wherein X is S. Also provided for each embodiment listed above, including those from and for Formulas (I) and Formulas (A-1) through (A-20), wherein X is —N(CH₃)—. Also provided for each embodiment listed above, including those from and for Formulas (I) and Formulas (A-1) through (A-20), wherein X is —C(CH₃)₂—. Also provided for each embodiment listed above, including those from and for Formulas (I) and Formulas (A-1) through (A-20), wherein X is —P(OOH)—. Also provided for each embodiment listed above, including those from and for Formulas (I) and Formulas (A-1) through (A-20), wherein X is —P(OPh)—. Also provided for each embodiment listed above, including those from and for Formulas (I) and Formulas (A-1) through (A-20), wherein X is —SO₂.

In one embodiment, provided herein is a compound of Formula (I):

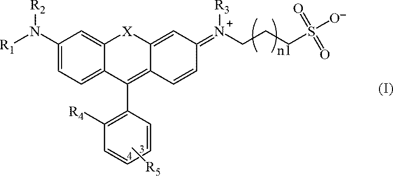

wherein:
X is selected from the group of O, —Si(CH₃)₂—, S, —N(CH₃)—, —C(CH₃)₂—, —P(OOH)—, —P(OPh)—, and —SO₂; R₁ and R₂ are independently selected from the group of H and C₁-C₃ alkyl, with the proviso that at least one of R₁ and R₂ is not H;
R₃ is selected from the group of H and C₁-C₃ alkyl;
R₄ is selected from the group of H and C₁-C₃ alkyl;
R₄ is selected from the group of H and C₁-C₃ alkyl;
R₅ is bound to either the 3-position carbon atom or the 4-position carbon atom of the phenyl ring and is selected from the group of H and

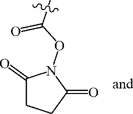

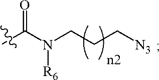

R₆ is selected from the group of H and C₁-C₃ alkyl;
n1 is an integer selected from the group of 0, 1, 2, and 3; and
n2 is an integer selected from the group of 0, 1, 2, and 3.

There is an additional embodiment comprising a compound of Formula (I), wherein R₅ is bound to the 3-position carbon atom of the phenyl ring and all other variables and definitions are as indicated for Formula (I), above.

There is also an additional embodiment comprising a compound of Formula (I), wherein R₅ is bound to the 4-position carbon atom of the phenyl ring and all other variables and definitions are as indicated for Formula (I), above.

There is another embodiment comprising a compound of Formula (I), wherein R₅ is bound to the 3-position carbon atom of the phenyl ring; R₄ is methyl; and all other variables and definitions are as indicated for Formula (I), above.

There is a further embodiment comprising a compound of Formula (I), wherein R₅ is bound to the 4-position carbon atom of the phenyl ring; R₄ is methyl; and all other variables and definitions are as indicated for Formula (I), above.

An embodiment provides a compound of Formula (la):

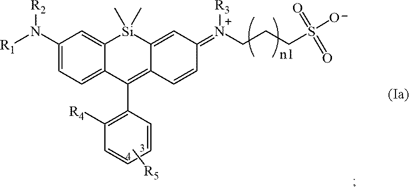

wherein R₁, R₂, R₃, R₄, R₅, R₆, n1, and n2 are as defined for Formula (I), above.

There is an additional embodiment comprising a compound of Formula (la), wherein R₅ is bound to the 3-position carbon atom of the phenyl ring and all other variables and definitions are as indicated for Formula (I), above.

There is also an additional embodiment comprising a compound of Formula (Ia), wherein R₅ is bound to the 4-position carbon atom of the phenyl ring and all other variables and definitions are as indicated for Formula (I), above.

There is another embodiment comprising a compound of Formula (Ia), wherein R₅ is bound to the 3-position carbon atom of the phenyl ring; R₄ is methyl; and all other variables and definitions are as indicated for Formula (I), above.

There is a further embodiment comprising a compound of Formula (la), wherein R₅ is bound to the 4-position carbon atom of the phenyl ring; R₄ is methyl; and all other variables and definitions are as indicated for Formula (I), above.

Another embodiment provides a compound of Formula (Ib):

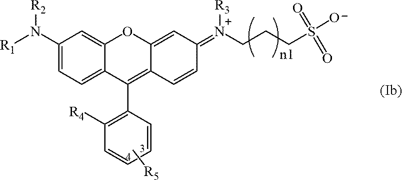

wherein R₁, R₂, R₃, R₄, R₅, R₆, n1, and n2 are as defined for Formula (I), above.

There is an additional embodiment comprising a compound of Formula (Ib), wherein R₅ is bound to the 3-position carbon atom of the phenyl ring and all other variables and definitions are as indicated for Formula (I), above.

There is also an additional embodiment comprising a compound of Formula (Ib), wherein R₅ is bound to the 4-position carbon atom of the phenyl ring and all other variables and definitions are as indicated for Formula (I), above.

There is another embodiment comprising a compound of Formula (Ib), wherein R₅ is bound to the 3-position carbon atom of the phenyl ring; R₄ is methyl; and all other variables and definitions are as indicated for Formula (I), above.

There is a further embodiment comprising a compound of Formula (Ib), wherein R₅ is bound to the 4-position carbon atom of the phenyl ring; R₄ is methyl; and all other variables and definitions are as indicated for Formula (I), above.

Another embodiment provides a compound of Formula (II):

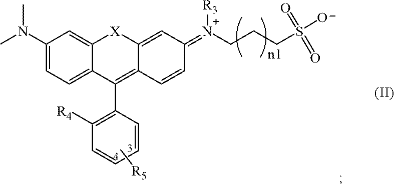

wherein each variable, including X, R₃, R₄, R₅, R₆, n1, and n2, are as defined for the compound of Formula (I), above.

There is an additional embodiment comprising a compound of Formula (II), wherein R₅ is bound to the 3-position carbon atom of the phenyl ring and all other variables and definitions are as indicated for Formula (I), above.

There is also an additional embodiment comprising a compound of Formula (II), wherein R₅ is bound to the 4-position carbon atom of the phenyl ring and all other variables and definitions are as indicated for Formula (I), above.

There is another embodiment comprising a compound of Formula (II), wherein R₅ is bound to the 3-position carbon atom of the phenyl ring; R₄ is methyl; and all other variables and definitions are as indicated for Formula (I), above.

There is a further embodiment comprising a compound of Formula (II), wherein R₅ is bound to the 4-position carbon atom of the phenyl ring; R₄ is methyl; and all other variables and definitions are as indicated for Formula (I), above.

Another embodiment provides a compound of Formula (IIa):

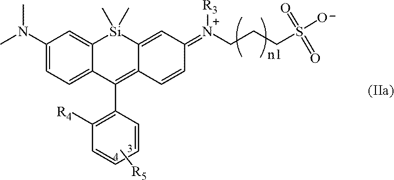

wherein each variable, including R₃, R₄, R₅, R₆, n1, and n2, are as defined for the compound of Formula (I), above.

There is an additional embodiment comprising a compound of Formula (IIa), wherein R₅ is bound to the 3-position carbon atom of the phenyl ring and all other variables and definitions are as indicated for Formula (I), above.

There is also an additional embodiment comprising a compound of Formula (IIa), wherein R₅ is bound to the 4-position carbon atom of the phenyl ring and all other variables and definitions are as indicated for Formula (I), above.

There is another embodiment comprising a compound of Formula (IIa), wherein R₅ is bound to the 3-position carbon atom of the phenyl ring; R₄ is methyl; and all other variables and definitions are as indicated for Formula (I), above.

There is a further embodiment comprising a compound of Formula (IIa), wherein R₅ is bound to the 4-position carbon atom of the phenyl ring; R₄ is methyl; and all other variables and definitions are as indicated for Formula (I), above.

A further embodiment provides a compound of Formula (IIb):

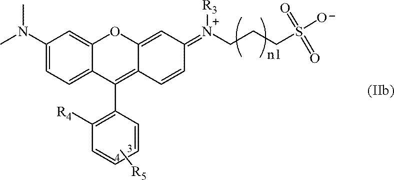

wherein each variable, including R₃, R₄, R₅, R₆, n1, and n2, are as defined for the compound of Formula (I), above.

There is an additional embodiment comprising a compound of Formula (IIb), wherein R₅ is bound to the 3-position carbon atom of the phenyl ring and all other variables and definitions are as indicated for Formula (I), above.

There is also an additional embodiment comprising a compound of Formula (IIb), wherein R₅ is bound to the 4-position carbon atom of the phenyl ring and all other variables and definitions are as indicated for Formula (I), above.

There is another embodiment comprising a compound of Formula (IIb), wherein R₅ is bound to the 3-position carbon atom of the phenyl ring; R₄ is methyl; and all other variables and definitions are as indicated for Formula (I), above.

There is a further embodiment comprising a compound of Formula (IIb), wherein R₅ is bound to the 4-position carbon atom of the phenyl ring; R₄ is methyl; and all other variables and definitions are as indicated for Formula (I), above.

A further embodiment provides a compound of Formula (III):

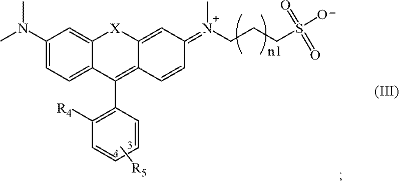

wherein each variable, including X, R₄, R₅, R₆, n1, and n2, are as defined for the compound of Formula (I), above.

There is an additional embodiment comprising a compound of Formula (III), wherein R₅ is bound to the 3-position carbon atom of the phenyl ring and all other variables and definitions are as indicated for Formula (I), above.

There is also an additional embodiment comprising a compound of Formula (III), wherein R₅ is bound to the 4-position carbon atom of the phenyl ring and all other variables and definitions are as indicated for Formula (I), above.

There is another embodiment comprising a compound of Formula (III), wherein R₅ is bound to the 3-position carbon atom of the phenyl ring; R₄ is methyl; and all other variables and definitions are as indicated for Formula (I), above.

There is a further embodiment comprising a compound of Formula (III), wherein R₅ is bound to the 4-position carbon atom of the phenyl ring; R₄ is methyl; and all other variables and definitions are as indicated for Formula (I), above.

Another embodiment provides a compound of Formula (IIIa):

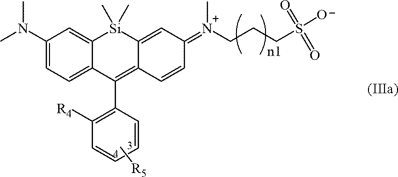

wherein each variable, including R₄, R₅, R₆, n1, and n2, are as defined for the compound of Formula (I), above.

There is an additional embodiment comprising a compound of Formula (Illa), wherein R₅ is bound to the 3-position carbon atom of the phenyl ring and all other variables and definitions are as indicated for Formula (I), above.

There is also an additional embodiment comprising a compound of Formula (Illa), wherein R₅ is bound to the 4-position carbon atom of the phenyl ring and all other variables and definitions are as indicated for Formula (I), above.

There is another embodiment comprising a compound of Formula (Illa), wherein R₅ is bound to the 3-position carbon atom of the phenyl ring; R₄ is methyl; and all other variables and definitions are as indicated for Formula (I), above.

There is a further embodiment comprising a compound of Formula (Illa), wherein R₅ is bound to the 4-position carbon atom of the phenyl ring; R₄ is methyl; and all other variables and definitions are as indicated for Formula (I), above.

Another embodiment provides a compound of Formula (IIIb):

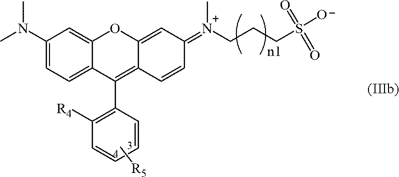

wherein each variable, including R₄, R₅, R₆, n1, and n2, are as defined for the compound of Formula (I), above.

There is an additional embodiment comprising a compound of Formula (Illb), wherein R₅ is bound to the 3-position carbon atom of the phenyl ring and all other variables and definitions are as indicated for Formula (I), above.

There is also an additional embodiment comprising a compound of Formula (IIIb), wherein R₅ is bound to the 4-position carbon atom of the phenyl ring and all other variables and definitions are as indicated for Formula (I), above.

There is another embodiment comprising a compound of Formula (IIIb), wherein R₅ is bound to the 3-position carbon atom of the phenyl ring; R₄ is methyl; and all other variables and definitions are as indicated for Formula (I), above.

There is a further embodiment comprising a compound of Formula (IIIb), wherein R₅ is bound to the 4-position carbon atom of the phenyl ring; R₄ is methyl; and all other variables and definitions are as indicated for Formula (I), above.

Another embodiment provides a compound of Formula (IV):

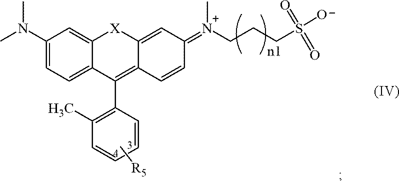

wherein each variable, including X, R₅, R₆, n1, and n2, are as defined for the compound of Formula (I), above.

There is an additional embodiment comprising a compound of Formula (IV), wherein R₅ is bound to the 3-position carbon atom of the phenyl ring and all other variables and definitions are as indicated for Formula (I), above.

There is also an additional embodiment comprising a compound of Formula (IV), wherein R₅ is bound to the 4-position carbon atom of the phenyl ring and all other variables and definitions are as indicated for Formula (I), above.

A further embodiment provides a compound of Formula (Iva):

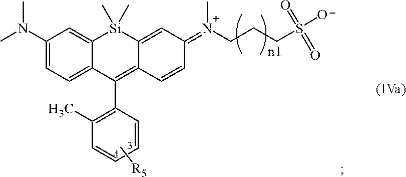

wherein each variable, including R₅, R₆, n1, and n2, are as defined for the compound of Formula (I), above.

There is an additional embodiment comprising a compound of Formula (IVa), wherein R₅ is bound to the 3-position carbon atom of the phenyl ring and all other variables and definitions are as indicated for Formula (I), above.

There is also an additional embodiment comprising a compound of Formula (IVa), wherein R₅ is bound to the 4-position carbon atom of the phenyl ring and all other variables and definitions are as indicated for Formula (I), above.

Another embodiment provides a compound of Formula (IVb):

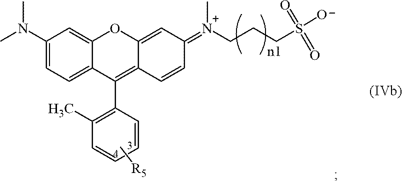

wherein each variable, including R₅, R₆, n1, and n2, are as defined for the compound of Formula (I), above.

There is an additional embodiment comprising a compound of Formula (IVb), wherein R₅ is bound to the 3-position carbon atom of the phenyl ring and all other variables and definitions are as indicated for Formula (I), above.

There is also an additional embodiment comprising a compound of Formula (IVb), wherein R₅ is bound to the 4-position carbon atom of the phenyl ring and all other variables and definitions are as indicated for Formula (I), above.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, R₆, and n2, are as defined for the compound of the embodiment in question, except that n1 is an integer selected from 0, 1, and 2.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, R₆, and n1 are as defined for the compound of the embodiment in question, except that n2 is an integer selected from 0, 1, and 2.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, and R₆ are as defined for the compound of the embodiment in question, except that n1 is an integer selected from 0, 1, and 2, and n2 is an integer selected from 0, 1, and 2.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, R₆, and n2, are as defined for the compound of the embodiment in question, except that n1 is 1.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, R₆, and n1 are as defined for the compound of the embodiment in question, except that n2 is 1.

Within each of the embodiments herein, including those for Formula (I), Formula (la), Formula (Ib), Formula (II), Formula (IIa), Formula (IIb), Formula (III), Formula (IIIa), Formula (IIIb ), Formula (IV), Formula (IVa), and Formula (IVb), above, there is a further embodiment wherein R₁, R₂, R₃, R₄, R₅, and R₆ are as defined for the compound of the embodiment in question, except that n1 is 1 and n2 is 1.

Also provided is a composition comprising an effective amount of a compound of Formula (I). Additional individual compositions are also provided, each comprising an effective amount, respectively, of a compound of Formula (la), Formula (Ib), Formula (II), Formula (IIa), Formula (IIb), Formula (III), Formula (Illa), Formula (IIIb ), Formula (IV), Formula (IVa), and Formula (IVb), or any of the individual compounds described herein.

Also provided herein is the use of a compound of Formula (I) in the preparation of a composition for use in fluorescence nanoscopy. Additional individual uses of a compound of Formula (la), Formula (Ib), Formula (II), Formula (IIa), Formula (IIb), Formula (III), Formula (IIIa), Formula (IIIb ), Formula (IV), Formula (IVa), and Formula (IVb), or any of the individual compounds described herein, respectively, in the preparation of a composition for use in fluorescence nanoscopy.

In some embodiments herein, when R5 is the group below, n2 is 3:

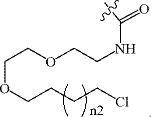

Also provided herein is a method of labeling a biomolecule, cell, tissue, or organ of interest, the method comprising contacting the biomolecule, cell, tissue, or organ of interest, with at least one fluorophore compound of interest, including a fluorophore selected from Formula (I) and/or the other formulas provided herein.

Labeling methods can involve contacting the biomolecules, cell, tissue, or organ of interest with at least one fluorophore compound described herein (such as those of Formulas (I), (II), (III), and (IV), under conditions suitable for labeling. Typically, the labeling will be performed in a liquid solution with other chemical agents present. The additional chemical agents can include salts, buffers, detergents, and so on. The liquid solution can also include water and/or other solvents such as methanol, ethanol, dimethylsulfoxide (DMSO), and tetrahydrofuran (THF).

The in vivo applications can involve contacting the fluorophore compound with cells suspended in culture, with cells immobilized on a surface, with a slice of tissue, with a monolayer of cells, with a tissue, or with an intact organism. For example, the fluorophore compounds may directly insert into the membrane of the cell or pass through the membrane into the cytoplasm. The in vivo applications can further comprise a step of enhancing the ability of the target cells to uptake the fluorophore compound. The enhancing step can comprise treating the cells with a detergent, treating the cells with dimethylsulfoxide (DMSO), treating the cells with one or more pulses of an electrical charge (electroporation), or treating the cells briefly with osmotic shock. Alternatively, the contacting step can comprise direct injection of the fluorophore compound into the cell using a micropipette or other syringe devices.

The liquid solution can generally be at any pH compatible with the biomolecule and the fluorophore compound. For example, the pH can be about 5, about 5.5, about 6, about 6.5, about 7, about 7.5, about 8, about 8.5, about 9, and ranges between any two of these values.

The liquid solution can generally be at any temperature compatible with the biomolecule and the fluorophore compound. Typically, the liquid solution will be at a temperature of about 0° C. to about 50° C. Temperatures can be about 0° C., about 5° C., about 10° C., about 15° C., about 20° C., about 25° C., about 30° C., about 35° C., about 40° C., about 45° C., about 50° C., and ranges between any two of these values.

The contacting step can generally be performed for any suitable length of time. For example, the contacting step can be performed for about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours or longer, or ranges between any two of these values.

The methods of use can further comprise a purification step performed after the contacting step. The purification step can comprise separating unbound fluorophore compound from fluorophore compound bound to the biomolecules. The purification step can comprise the use of chromatography (such as agarose gel electrophoresis, polyacrylamide gel electrophoresis (“PAGE”), SDS-polyacrylamide gel electrophoresis (“SDS-PAGE”), isoelectric focusing, affinity chromatography, size-exclusion chromatography, separation with magnetic particles, ELISA, HPLC, FPLC, centrifugation, density gradient centrifugation, dialysis, or osmosis.

The methods of use can further comprise visualizing the fluorophore compound bound to the biomolecules. The visualization can be performed by illumination by a light source followed by epifluorescence microscopy, by total internal reflection fluorescence microscopy, by confocal microscopy, by two-photon or multi-photon excitation microscopy, by second-harmonic imaging microscopy, by polarization microscopy, or by aperture-based or apertureless near-field optical microscopy. The methods of use can further comprise quantifying the fluorophore compound bound to the biomolecules. The quantification can be performed by counting detected photons in a time interval, by pumping the fluorophore with light of different polarizations, by measuring the polarization of the detected photons, by measuring the anisotropy of the detected photons, by measuring the spectrum of the detected photons, by measuring the lifetime of the detected photons, or by measuring the correlations of the detected photons. Correlations can be measured by fluorescence correlation spectroscopy, by start-stop coincidence counting, by using hardware autocorrelators, or by time-tagging the emission time of each photon with respect to the time of a pumping light pulse followed by off-line computation.

As such, provided is a method of conjugation or bioconjugation, the method comprising forming at least one covalent bond between a compound of Formula (I) and a biological molecule, such as a biological molecule selected from the group of a peptide, protein, carbohydrate, nucleic acid, toxin, or lipid. One method provides a method of conjugation or bioconjugation, the method comprising forming at least one covalent bond between a compound of Formula (I) and an antibody.

Methods of using the compounds herein or their conjugates or bioconjugates as such or after photoactivation as labels in optical microscopy and imaging techniques, microfluidic devices, capillary electrophoresis, or protein tracking techniques comprise labelling an object (such as a biological molecule) with the compounds or their conjugates and detecting the objects in an optical microscope, an imaging device, a microfluidic device, a capillary electrophoresis device or a protein tracking device.

The methods of use herein include those wherein the optical microscopy and imaging techniques are selected from the group consisting of stimulated emission depletion microscopy (STED), single molecule switching (SMS) “nanoscopy” (diffraction unlimited optical resolution by using switching of the fluorescence of the single molecules), fluorescence correlation spectroscopy (FCS), fluorescence recovery after photobleaching (FRAP), fluorescence lifetime imaging (FLIM), ground state depletion with individual molecular return (GSDIM), fluorescence resonant energy transfer (FRET), and reversible saturable/switchable optical linear (fluorescence) transitions (RESOLFT) methods and equipment.

### Definitions

The terms “fluorescence nanoscopy” and “super-resolution microscopy” refer to optical imaging techniques to reach the nanometer resolution range, typically 20-50 nm and even down to the 1 nm level. These nanoscopic techniques enable not only cell surface examination, but also examination of the entire cell interior at levels of detail previously not available with light microscopy.

The term “alkyl” refers to a straight or branched hydrocarbon. For example, an alkyl group can have 1 to 23 carbon atoms (i.e, C₁-C₃ alkyl or C₁₋₃ alkyl), 1 to 2 carbon atoms (i.e., C₁-C₂ alkyl or C₁₋₂ alkyl). A C₁-C₃ alkyl group is understood to include methyl, ethyl, n-propyl, and isopropyl alkyl groups.

As used herein, the terms “azide,” “azido,” “N3,” “-N3,” “N-3,” “-N-3,” “-N=N+=N-,” “-N-=N+ΞN,” and the moieties:

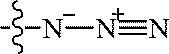

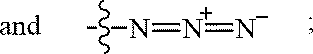

and the like, each refer to a substituent group that is the conjugate base of hydrazoic acid.

The terms “variable” and “variables” refer to one or more groups in a chemical description or structure that may vary in definition. Non-limiting examples, for instance, include “R” groups or other substituents (such as R₁, R₂, R₃, R₄ₐ, R_{4b}, R₅, etc.), integers (such as n1 and n2), ring atoms (such as “X”), and the like.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

The terms “UV-Vis” or “UV/Vis” refer to ultraviolet-visible spectroscopy or ultraviolet-visible spectrophotometry, which are absorption spectroscopy or reflectance spectroscopy in part of the ultraviolet and the full, adjacent visible regions of the electromagnetic spectrum.

As used herein, the terms “effective amount” or “diagnostically effective amount” or “imaging effective amount” or the like refer to the quantity of a targeting construct necessary to aid in direct visualization of any target molecules, cell(s), tissue(s), and/or organ(s) located in the body part under investigation in a subject or a biological sample.

Also provided herein is a pharmaceutical composition comprising an effective amount of a compound of Formula (I) and a pharmaceutically acceptable carrier or excipient.

The compounds provided herein may be used in methods, concentrations, and formulations known in the art for imaging agents. In systemic administration, the compounds may administered in a range of from about 0.05 mg/ml to about 2 mg/ml. In some embodiments, they may be administered at a concentration of from about 0.1 mg/ml to about 1.0 mg/ml. In some embodiments, the total injection volume may range from about 10 µl to about 300 µl. In other embodiments, the total injection volume may range from about 50 µl to about 200 µl.

In some embodiments the compounds herein and/or compositions comprising them are intended for direct/topical administration. Direct or topical administration are understood herein to comprise the administration of an agent or composition directly to surface of a sample, tissue, organ, other bodily component. In some methods, the administration may be accomplished by brushing, spraying, or irrigation with the appropriate compound or composition.

In other embodiments, the agents and/or compositions may be administered systemically to the patient or subject, such as through intravenous injection or infusion.

Parenteral formulations may comprise injectable fluids that are pharmaceutically and physiologically acceptable fluid carrier vehicles such as water, physiological and/or buffered saline, other balanced salt solutions, aqueous dextrose, glycerol or the like. Excipients may include, for example, nonionic solubilizers, such as polyethoxylated castor oil, or proteins, such as human serum albumin or plasma preparations. If desired, the pharmaceutical composition to be administered may also contain non-toxic auxiliary substances, such as wetting or emulsifying agents, preservatives, and pH buffering agents and the like, for example, sodium acetate or sorbitan monolaurate.

Useful injectable preparations include sterile suspensions, solutions or emulsions of the conjugate(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. The composition may take such forms as suspension, solutions or emulsions in oily or aqueous vehicles, and may contain formulatory agents such as suspending, stabilizing and/or dispersing agents. For example, parenteral administration may be done by bolus injection or continuous infusion. Alternatively, the conjugate may be in powder form for reconstitution with a suitable vehicle, e.g. sterile water, before use.

Systemic formulations include those designed for administration by injection, e.g., subcutaneous, intravenous, intramuscular, intrathecal or intraperitoneal injection, as well as those designed for transdermal, transmucosal, oral or pulmonary administration.

Formulations for topical administration may also comprise vehicles or carriers known in the art. Non-limiting examples include use of individual agents, or combinations thereof, selected from topical bases (such as polyethylene glycols), antioxidants (such as butylated hydroxyanisole, butylated hydroxytoluene, ascorbic acid, a tocopherol and combinations thereof), emollients (such as water, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl® 424 (myristyl myristate), octyl dodecanol, dimethicone (Dow Corning 200-100 cps), phenyl trimethicone (Dow Coming 556), Dow Coming 1401 (cyclomethicone and dimethiconol), and cyclomethicone (Dow Coming 344), and Miglyol® 840 (manufactured by Huls; propylene glycol dicaprylate/dicaprate)), penetration enhancers (such as dimethyl isosorbide, propylene glycol, or a combination thereof), water-miscible solvents, surfactants (such as sorbitan monostearate, a polyethylene glycol monostearate, D-a-tocopheryl polyethylene glycol 1000 succinate, a composition comprising glycol stearate / PEG32 stearate / PEG6 stearate, or a combination thereof), absorbents (such as hydrogels), astringents (such as witch hazel, alcohol, and herbal extracts such as chamomile extract), binders (such as starch, cellulose ethers, microcrystalline cellulose, calcium hydrogen phosphate, calcium phosphate dibasic, and calcium sulfate dihydrate), other excipients (such as polyvinylpyrrolidone (PVP), tocopheryl polyethylene glycol 1000 succinate (vitamin E TPGS, or TPGS), dipalmitoyl phosphatidyl choline (DPPC), trehalose, sodium bicarbonate, glycine, sodium citrate, and lactose), buffering agents (such as monobasic or dibasic potassium phosphate, monobasic or dibasic sodium phosphate, magnesium hydroxide), chelating agents (such as EDTA (ethylenediaminetetraacetic acid, tetrasodium salt)), film-forming agents (such as chitosan, hydroxypropylmethylcellulose, polyvinyl alcohol), conditioning agents (such as petrolatum, glycerin, propylene glycol), and opacifying agents (such as titanium dioxide), and pH adjusters (such as citric acid and sodium hydroxide).

A “subject” or a “patient” refers to any animal. The animal may be a mammal. Examples of suitable mammals include human and non-human primates, dogs, cats, sheep, cows, pigs, horses, mice, rats, rabbits, and guinea pigs. In some embodiments the subject or patient is a human, particularly including a human undergoing or in need of a surgical procedure or examination.

The compounds of the present invention may be prepared by methods known in the art, as well as those described below.

### General

All reagents were purchased from Sigma Aldrich, Fisher Scientific, or TCI. Unless otherwise indicated, all commercially available starting materials were used directly without further purification. Analytical TLC was performed on Millipore ready-to-use plates with silica gel 60 (F254, 32-63 µm). Purification was performed on a Biotage Isolera Flash System using pre-packed silica gel cartridges or on a reverse phase preparative HPLC (Agilent 1250 Infinity HPLC).

### Water Solubility Measurements

Each screening candidate was dissolved in a 1 mL mixture of chloroform and methanol (equal volume) with final stock concentrations ranging from 10 to 50 mM. The solvent was then removed in vacuo before 200 µL of DI water was added. The test sample was then vortexed before sonicated in an ultrasonic bath for 30 minutes. The undissolved pellet was removed by centrifugation at 13,000 rpm for 5 minutes. The supernatant was sampled and diluted with water before measured for absorbance using a SpectraMax M5 spectrometer with a Microplate reader (Molecular Devices, Sunnyvale, CA). The water solubility of each screening candidate was then calculated using Beer’s Law plots of absorbance versus concentration.

### Abbreviations

Abbreviations and acronyms used herein may include: Abs = Absorbance; anhy. = anhydrous; Boc = tert-butoxycarbonyl protecting group; CDX = Cell derived xenograft; DCE = Dichloroethane; DCM = Dichloromethane; DDQ = 2,3-Dichloro-5,6-dicyano-1,4-benzoquinone; DMF = Dimethylformamide; DMSO = Dimethylsulfoxide; DiPEA = N,N-Diisopropylethylamine; EDAC = N-(3-Dimethylaminopropyl)-N′-ethylcarbodiimide hydrochloride; equiv. = equivalent(s); EtOH = Ethanol; EtOAc = Ethyl acetate; Et₂O = Diethyl ether; Fl = Fluorescence; HATU = 1-[Bis(dimethylamino)methylene]-1H-1,2,3-triazolo[4,5-b]pyridinium 3-oxid hexafluorophosphate; HOBt = 1-Hydroxybenzotriazole hydrate; HPLC = High-performance liquid chromatography; LCMS = Liquid chromatography-mass spectrometry; MeCN = Acetonitrile; MeOH = Methanol; NMR = Nuclear magnetic resonance; Pd(PPh₃)₄ = tetrakis(triphenylphosphine)palladium; PBS = Phosphate buffered saline; PyBOP = (Benzo triazol-1-yloxy)tripyrrolidino phosphonium hexafluorophosphate; RT or rt = Room temperature; SD = Standard deviation; TFA = trifluoroacetic acid; THF = Tetrahydrofuran; TLC = Thin layer chromatography; and TSTU = O-(N-Succinimidyl)-N,N,N′,N′-tetramethyluronium tetrafluoroborate.

### LCMS Characterization

Mass-to-charge ratio and purity of the sulfonated rhodamine compounds were characterized on an Agilent 6244 time-of-flight tandem liquid chromatography mass spectroscopy (LCMS) with diode array detector VL+. Sample (10 µL) was injected into a C18 column (Poroshell 120, 4.6 × 50 mm, 2.7 micron), and eluted with a solvent system of A (H₂O, 0.1% FA) and B (MeCN, 01.% FA) at 0.4 mL/min, from A/B = 90/10 to 5/95 over 10 min, maintained at A/B = 5/95 for additional 5 min. Ions were detected in positive ion mode by setting the capillary voltage at 4 kV and gas temperature at 350° C. Purity was calculated through area under the curve analysis of the absorbance at 254 nm.

### UV-Vis Absorption and Fluorescence Spectroscopy

UV-Vis and fluorescence spectra were collected on a SpectraMax M5 spectrometer with a Microplate reader (Molecular Devices, Sunnyvale, CA). All absorbance spectra were reference corrected. Extinction coefficient was calculated from Beer’s Law plots of absorbance versus concentration. Relative quantum yields are reported using TMR and SiTMR as reference.

### Synthetic Details of Sulfonated Rhodamine Derivatives:

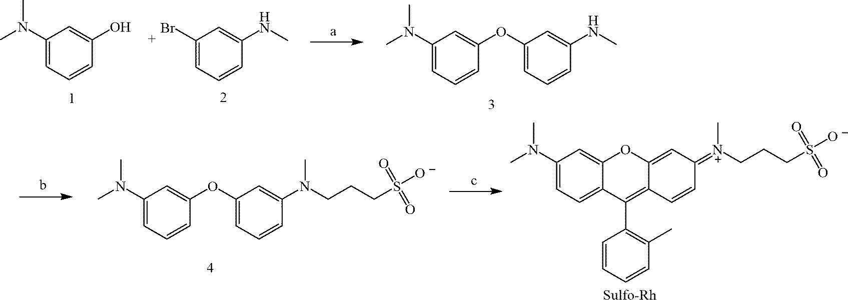

Scheme 1 - Synthetic route to Sulfo-Rh: Reagents and conditions: a) Cul, 2-picolinic acid, K₃PO₄, DMSO, 85° C.; b) Na₂CO₃, 1,3-propanesultone, MeCN, 85° C.; c) i) ZnCl₂, 2-methylbenzaldehyde, EtOH, 110° C.; ii) DDQ, DCM/MeOH, 0° C.

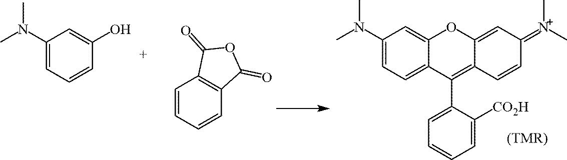

N-(2-carboxyphenyl)-6-(dimethylamino)-3H-xanthen-3-ylidene)-N-methylmethanaminium (TMR): In a 500 mL round-bottom flask, 3-(dimethylamino)phenol (7.5 g, 54.7 mmol, 1.02 equiv.) and isobenzofuran-1,3-dione (7.94 g, 53.6 mmol, 1.0 equiv.) were suspended in chlorobenzene (100 mL). Thereaction was placed on an oil bath, heated to 160° C., and refluxed for ca. 1 h. Additional 3-(dimethylamino)phenol (7.5 g, 54.7 mmol, 1.02 equiv.) was added to the reaction over 30 minutes. The bath temperature was kept at 150° C., and reaction mixture was stirred at this temperature overnight. Upon cooling down to rt, the reaction mixture was diluted with 1 M NaOH solution (40 mL), and stirred for an additional 30 minutes. The solvents were removed via decantation prior to the addition of 50% NaOH solution (100 mL). The resulting precipitate was collected via vacuum filtration and subsequently resuspended in DI water (150 mL), followed by addition of concentration H₂SO₄ (7.5 mL). The reaction mixture was then stirred for 30 minutes before concentrated HCl (15 mL) and NaCl (7.5 g) was added. The reaction was then heated to 60° C. and stirred at this temperature for 1 hour. Upon cooling to rt, the product was collected via vacuum filtration, washed with 2 % HCl (100 mL), air dried in the funnel overnight, affording pure TMR (6.62 g, 32%) as a green solid. HRMS(ESI) [M+H]⁺ m/z found 387.1764, calcd for C₂₄H₂₃N₂O₃ 387.1703.

### N,N-dimethyl-3-(3-(methylamino)phenoxy)aniline (3)

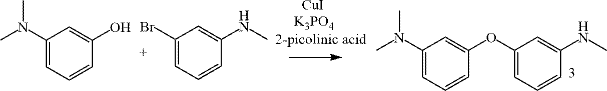

Compound 3 was synthesized according to a protocol modified slightly from that published by Maiti and Buchwald.¹⁹ A oven-dried heavy wall round bottom flask was charged with a magnetic stir bar, 3-(dimethylamino)phenol (1) (1.00 g, 7.29 mmol), 3-bromo-N-methylaniline (2) (1.42 g, 7.65 mmol), Cul (139 mg, 0.729 mmol), 2-picolinic acid (180 mg, 1.46 mmol), and anhydrous K₃PO₄ (3.09 g, 14.6 mmol). The flask was evacuated under vacuum and backfilled with N₂ (5x), then promptly sealed with a Teflon cap. Anhydrous DMSO (8 mL) was added and the reaction heated to 85° C. and left to stir for 24 h. Once cooled to room temperature (rt), the reaction mixture was diluted with 50 mL DI water and extracted with DCM (4 × 50 mL). The combined organic layers were washed with brine, dried over anhydrous Na₂SO₄, and concentrated in vacuo. The residue was purified by flash column chromatography in silica gel with DCM/Hexanes as eluent to give compound 3 (703 mg, 40%) as a colorless oil. HRMS(ESI) [M+H]⁺ m/z found 243.1492, calcd for C₁₅H₁₉N₂O 243.1492.

### 3-((Dimethylamino)phenoxy)phenyl)(methyl)amino)propane-1-sulfonate (4)

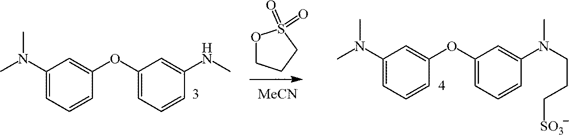

A 100 mL round-bottom flask was charged with N,N-dimethyl-3-(3-(methylamino)phenoxy)aniline (3) (0.6 g, 2.48 mmol, 1.0 equiv.) and a magnetic stir bar, then purged under N₂. The compound was then suspended in 35 mL anhydrous MeCN before 1,3-propanesultone (326 µL, 3.71 mmol, 1.5 equiv.) was added in a single portion. The reaction was placed on an oil bath, heated to 83° C., and refluxed for ca. 24 h. Afterwards, the reaction was cooled to rt prior to addition of an additional 2.5 equivalents sultone. The reaction was then stirred at reflux for an additional 24 h, before cooling to rt, diluting with 50 mL DCM, and filtering through a pre-packed Celite funnel. Organic solvent was removed under reduced pressure to give the product 4 as a highly viscous blue oil. Subsequent purification by reverse phase HPLC with mobile phase of MeCN/H₂O (gradient, 1-70% of MeCN in H₂O) afforded the product (629 mg, 70%). HRMS(ESI) [M+H]⁺ m/z found 365.1568, calcd for C₁₈H₂₅N₂O₄S 365.1530.

### E)((6-(dimethylamino)-9-(o-tolyl)-3H-xanthenylidene)(methyl)ammonio)propane-1-sulfonate (Sulfo-Rh, OF550)

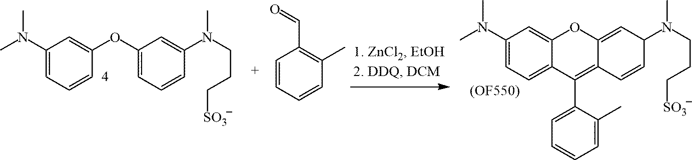

An oven-dried 100 mL round-bottom flask was charged with a magnetic stir bar and purged under N₂ for ca. 30 minutes. Compound (4) (200 mg, 0.518 mmol) and 2-methylbenzaldehyde (62 mg, 0.518 mmol) were transferred via syringe and suspended with anhydrous EtOH (20 mL). To the mixture was added anhydrous ZnCl₂ (2.12 g, 15.5 mmol) rapidly, in one portion. The flask was placed on an oil bath, heated to 110° C., and refluxed for 8 hours. The reaction was then removed from the oil bath. The residue was suspended in 20 mL DCM/MeOH (3:1) and chilled in an ice bath for 15 minutes before DDQ (176 mg, 0.776 mmol) was slowly added to the mixture. This was allowed to stir at 0° C. for another 15 minutes before organic solvent was removed under reduced pressure. The residue was purified on a Biotage Isolera Flash System using a SNAP Ultra cartridge with a mobile phase of DCM and MeOH containing 5% MeCN and 1% formic acid (gradient, 5-30% of MeOH in DCM). The title compound was obtained (79.8 mg, 33%) as a dark red solid. HRMS(ESI) [M+H]⁺ m/z found 465.1879, calcd for C₂₆H₂₉N₂O₄S 465.1843.

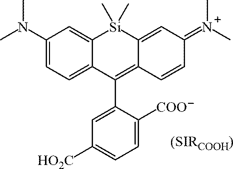

4-carboxy-2-(7-(dimethylamino)-3-(dimethyliminio)-5,5-dimethyl-3,5-dihydrodibenzo[b,e]silin-10-yl)benzoate (5, SiR_{COOH}): SiR_{COOH} was prepared following previously reported protocol. (Lukinavičius et al., Nature Chemistry 5, 132-139 (2013)).

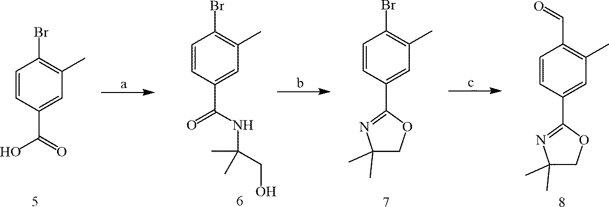

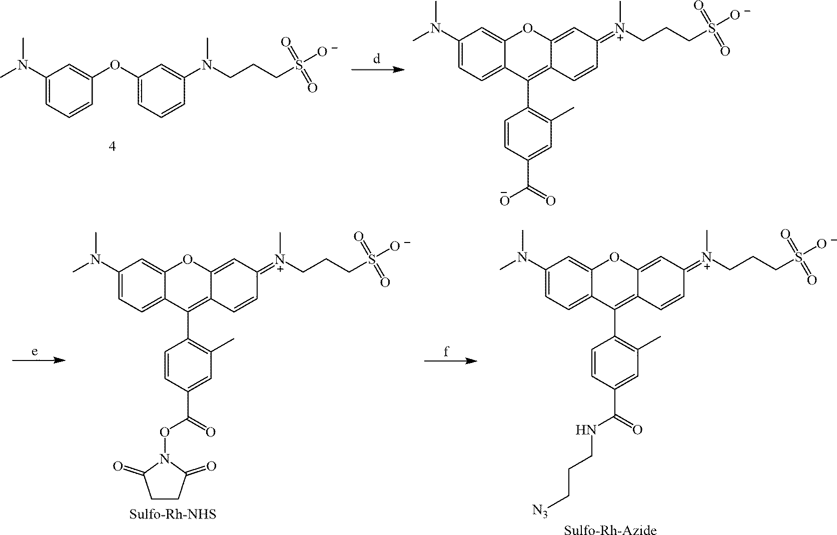

Scheme 2 - Synthetic route to reactive sulfonated rhodamines Sulfo-Rh-NHS and Sulfo-Rh-Azide: Reagents and conditions: a) i) thionyl chloride (SOCl₂), 70° C.; ii) DCM, 2-amino-2-methylpropan-1-ol, diisopropylethylamine (DiPEA), rt; b) SOCl₂, rt; c) i) n-butyllithium (n-BuLi), DMF, -78° C. to rt; d) i) Compounds 8, ZnCl₂, EtOH, 110° C.; ii) 6 M HCl, 80° C.; iii) 4,5-dichloro-3,6-dioxocyclohexa-1,4-diene-1,2-dicarbonitrile (DDQ), DCM/MeOH, 0° C.; e) N,N,N′,N′-tetramethyl-O-(N-succinimidyl)uronium tetrafluoroborate (TSTU), DiPEA, DMF, rt; f) 3-azido-1-propanamine, DiPEA, DMF, rt.

### 4-bromo-N-(1-hydroxy-2-methylpropan-2-yl)-3-methylbenzamide (6)

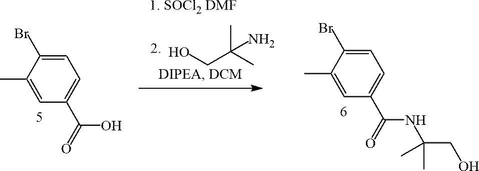

A 250 mL round-bottom flask was charged with 4-bromo-3-methylbenzoic acid 5 (10 g, 45.5 mmol, 1.0 equiv), a magnetic stir bar, and purged under N₂ for ca. 20 minutes before SOCl₂ (7.5 equiv., 346.78 mmol) was added. A catalytic amount of anhy. DMF (0.15 equiv., 6.98 µmol) was added before refluxing at ca. 75° C. for 3 hours. The reaction mixture was cooled to rt before SOCl₂ was removed under reduced pressure, and the residue re-dissolved in 50 mL DCM. The resultant mixture was then added dropwise (30 mL/h) to a solution of 2-amino-2-methylpropan-1-ol (1.25 equiv., 58.13 mmol) in 50 mL DCM containing DiPEA (1.25 equiv., 58.13 mmol) and stirred overnight at rt. Following O/N stirring, DCM was removed under reduced pressure, the residue was suspended in 50 mL of saturated NaHCO₃ solution and extracted with ethyl acetate (4× 50 mL). Combined organic layers were then washed with 5% LiCl solution (2× 100 mL), dried over anhydrous Na₂SO₄, filtered, and concentrated in vacuo to give (6) as a crude brown oil, used in the next step without further purification. HRMS(ESI) [M+H]⁺ m/z found 288.0417, calcd for C₁₂H₁₇BrNO₂ 288.0420.

### 2-Bromo-3-methylphenyl)-4,4-dimethyl-4,5-dihydrooxazole (7)

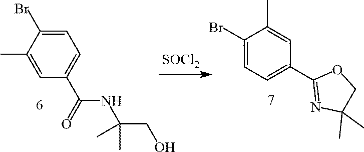

Compound (6) (ca. 14 g, 48.9 mmol) was transferred to an oven-dried round-bottom flask with DCM and purged with N₂ for ca. 30 minutes. The crude oil was then treated with 6 molar equivalents of SOCl₂ (28.9 mL, 398.37 mmol) and stirred at rt for 1 hour. SOCl₃ was then removed under reduced pressure, and the residue neutralized by the careful addition of 50 mL saturated NaHCO₃ solution. The aqueous layer was extracted with DCM (3× 50 mL), and the organic layers were combined, dried over anhydrous Na₂SO₄, filtered, and concentrated in vacuo. The crude oil was purified by column chromatography with DCM containing 1% MeCN used as eluent to afford (7) (7.42 g, 61%) as a fluid, amber oil. The reaction was repeated twice to provide sufficient materials for the subsequent reactions. HRMS(ESI) [M+H]⁺ m/z found 268.0345, calcd for C₁₂H₁₅BrNO 268.0332.

### 4-(4,4-Dimethyl-4,5-dihydrooxazol-2-yl)-2-methylbenzaldehyde (8)

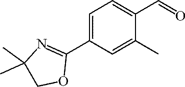 [SMILES: Cc1cc(C2=NC(C)(C)CO2)ccc1C=O]

A flame-dried 100 mL round-bottom flask was charged with a magnetic stir bar and purged under N₂ for ca. 30 minutes prior to the addition of (7) (2.00 g, 7.46 mmol) as a solution in anhy. THF (20 mL). The solution was then chilled to ca. -78° C. in a bath of dry ice in acetone over 30 minutes under N₂ before n-BuLi (1.2 equiv., 1.6 M in hexanes, 8.95 mmol) solution was added dropwise over ca. 20 minutes. The resultant mixture was then stirred at -78° C. for 45 minutes before anhy. DMF (1.4 equiv., 10.44 mmol) was added in one portion and the mixture stirred for an additional hour at -78° C. The reaction was then removed from the cold bath and stirred at rt for 1 hour before excess BuLi was quenched by the addition of 25 mL DI water. The aqueous layer was then extracted with diethyl ether (Et₂O, 1× 50 mL), followed by DCM (3× 50 mL). Combined organic layers were dried over anhy. Na₂SO₄, filtered, and concentrated in vacuo. Purification by column chromatography with EtOAc/Hexanes (⅕) as eluent to afford product 8 (1.42 g, 88%) as an oil. HRMS(ESI) [M+H]⁺ m/z found 218.1127, calcd for C₁₃H₁₆NO₂ 218.1176.

### E)((9-(4-carboxy-2-methylphenyl)-6-(dimethylamino)-3H-xanthenylidene)(methyl)ammonio) propane-1-sulfonate (8, OF550_{COOH})

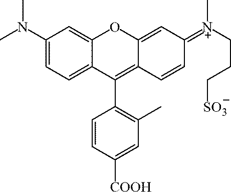

A flame-dried 100 mL round-bottom flask was charged with a magnetic stir bar and purged under N₂ for ca. 30 minutes. Compounds 4 (348 mg, 0.921 mmol) and 8 (200 mg, 0.921 mmol) were suspended in anhydrous EtOH (20 mL). To the mixture was added anhydrous ZnCl₂ (1.88 g, 13.8 mmol) rapidly, in one portion. The flask was placed on an oil bath, heated to 110° C., and refluxed for 8 hours. The solvent was removed under reduced pressure, and the residue was then resuspended in 6 M HCl (10 mL). The resulting mixture was heated at 78° C. and stirred overnight. The reaction was then removed from the oil bath and cooled to rt, then deposited onto a C18 cartridge, which was eluted with DI water followed by MeCN. The solution was concentrated in vacuo and the residue was suspended in 20 mL DCM/MeOH (3:1). The resulting solution was then chilled in an ice bath for 15 minutes before DDQ (1.5 equiv.) was slowly added to the mixture. This was allowed to stir at 0° C. for another 15 minutes before organic solvent was removed under reduced pressure. The residue was purified on a Biotage Isolera Flash System using a SNAP Ultra cartridge with a mobile phase of DCM and MeOH containing 5% MeCN and 1% formic acid (gradient, 5-30% of MeOH in DCM). The fractions containing the product were pooled and contracted in vacuo. Final purification was completed on a prep-HPLC system.

### E)((6-(dimethylamino)-9-(4-(((2,5-dioxopyrrolidin-1-yl)oxy)carbonyl)-2-methylphenyl)-3H-xanthenylidene)(methyl)ammonio)propane-1-sulfonate (Sulfo-Rh-NHS)

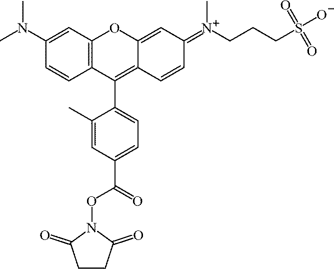 [SMILES: Cc1cc(C(=O)ON2C(=O)CCC2=O)ccc1-c1c2cc/c(=[N+](/C)CCCS(=O)(=O)[O-])cc-2oc2cc(N(C)C)ccc12]

Under N₂, a scintillation vial was charged with compound 9 (75 mg, 0.148 mmol) and TSTU (67 mmg, 0.221 mmol). The vial was purged under N₂ for an additional 15 min before adding 1 mL anhydrous DMF, followed by DiPEA (77 µL, 0.442 mmol). The reaction mixture was covered from light and stirred at rt for 1 h. The crude product was purified by reverse phase prep-HPLC (10-90% MeCN/H₂O gradient with 0.1% TFA), afforded the title compound (43 mg, 48%) as a dark red solid. HRMS(ESI) [M+H]⁺ m/z found 606.1907, calcd for C₃₁H₃₂N₃O₈S 606.1905.

### E)((9-(4-((3-azidopropyl)carbamoyl)-2-methylphenyl)-6-(dimethylamino)-3H-xanthenylidene)(methyl)ammonio)propane-1-sulfonate (Sulfo-Rh-Azide)

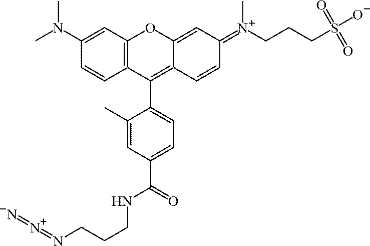 [SMILES: Cc1cc(C(=O)NCCCN=[N+]=[N-])ccc1-c1c2cc/c(=[N+](/C)CCCS(=O)(=O)[O-])cc-2oc2cc(N(C)C)ccc12]

Under N₂, a scintillation vial was charged with compound Sulfo-Rh-NHS (20 mg, 0.033 mmol) and 3-azido-1-propanamine (6.6 mg, 0.066 mmol). The vial was purged under N₂ for an additional 15 min before adding 0.5 mL anhydrous DMF, followed by DiPEA (17 µL, 0.099 mmol). The reaction mixture was covered from light and stirred at rt for 12 h. The crude product was purified by reverse phase prep-HPLC (10-90% MeCN/H₂O gradient with 0.1% TFA), afforded the title compound (17 mg, 87%) as a dark red solid. HRMS(ESI) [M+H]⁺ m/z found 513.2269, calcd for C₂₈H₂₉N₆O₄ 513.2245.

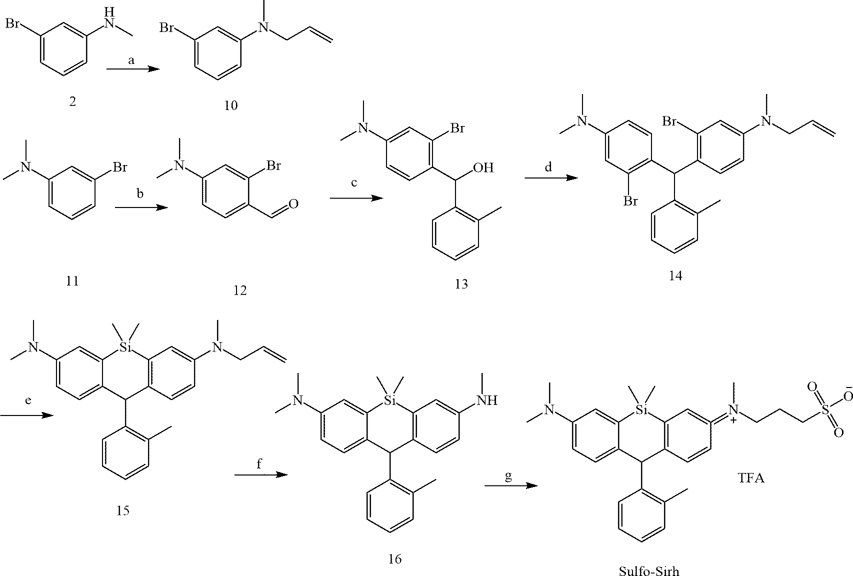

Scheme 3 - Synthetic route to Sulfo-SiRh: Reagents and conditions: a) Allyl bromide, Na₂CO₃, MeCN, 85° C.; b) i) Compound 11, Dichloroethane (DCE), DMF, phosphorus(V) oxychloride (POCl₃), 85° C.; c) i) 2-bromotoluene, DMF, n-BuLi, -78° C. to rt; d) Compound 10, DCM, ZnCl₂, 34° C.; e) i) t-BuLi, THF, -78° C.; ii) dichlorodimethylsilane, -78° C. to rt; f) Pd(PPh₃)₄, dimethylbarbituric acid (DMBA), DCM, 35° C.; g) i) 1,3-propanesultone, MeCN, 85° C.; ii) DDQ, DCM/MeOH, 0° C.

### N-allylbromo-N-methylaniline (10)

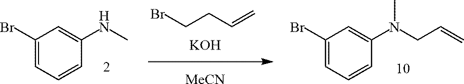

1) A 100 mL round-bottom flask was charged with compound 2 (2.00 g, 10.75 mmol) and ground KOH (1.21 g, 21.5 mmol), a magnetic stir bar, capped with rubber stopper and purged with N₂ for ca. 15 minutes. Reagents were suspended in 30 mL anhydrous MeCN prior to addition of allyl bromide (2.90 g, 21.5 mmol) in one portion, whereupon the reaction was placed on an oil bath, heated to 83° C., and refluxed overnight. Additional allyl bromide (2 equiv., 21.5 mmol) was added after overnight reflux to push the reaction further towards completion. Once bromoaniline had been consumed (determined by TLC) the reaction mixture was cooled to rt and diluted with DCM prior to filtration through a pre-packed celite funnel, then concentrated in vacuo. Purification by flash column chromatography with a gradient of EtOAc (1-10%) in hexanes used as eluent gave the product as a translucent, bronze colored oil (1.98 g, 82%) HRMS(ESI) [M+H]⁺ m/z found 226.0201, calcd for C₁₀H₁₃BrN 226.0226.

2) N-allyl-3-bromo-N-methylaniline (10): A 100 mL round-bottom flask was charged with compound 2 (10.0 g, 53.8 mmol, 1.0 equiv.), ground KOH (6.03 g, 108 mmol, 2.0 equiv.), a magnetic stir bar, then capped with a rubber stopper and purged with N₂ for ca. 15 minutes. Reagents were suspended in 50 mL anhydrous MeCN prior to addition of allyl bromide (10.4 mL, 108 mmol, 2.0 equiv.) in one portion, whereupon the reaction was placed on an oil bath, heated to ca. 83° C., and refluxed overnight. Additional allyl bromide (10.4 mL, 108 mmol, 2.0 equiv.) was added after overnight reflux to push the reaction further towards completion. Once bromoaniline had been consumed (determined by HPLC-MS), the reaction mixture was cooled to rt and diluted with DCM prior to filtration through a pre-packed celite filter funnel, then concentrated in vacuo. Purification by column chromatography with a gradient of EtOAc (1-10%) in hexanes used as eluent gave N-allyl-3-bromo-N-methylaniline as a translucent, bronze colored oil (10.2 g, 84%). HRMS(ESI) [M+H]⁺ m/z found 226.0201, calcd for C₁₀H₁₃BrN 226.0226.

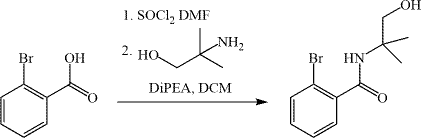

2-bromo-N-(1-hydroxy-2-methylpropan-2-yl)benzamide: A 250 mL round-bottom flask was charged with 2-bromobenzoic acid (10.0 g, 49.8 mmol, 1.0 equiv.), a magnetic stir bar, and purged under N₂ for ca. 20 minutes before thionyl chloride (SOCl₂, 20 mL) was added. A catalytic amount of anhy. N,N-dimethylformamide (DMF, 1 mL) was added before refluxing at ca. 75° C. for 3 hours. The reaction mixture was cooled to room temperature (rt) before SOCl₂ was removed under reduced pressure, and the residue re-dissolved in 50 mL dichloromethane (DCM). The resultant mixture was then added dropwise (30 mL/h) to a solution of 2-amino-2-methylpropan-1-ol (5.93 mL, 1.25 equiv., 62.2 mmol) in 100 mL DCM containing DiPEA (20 mL) and stirred overnight at rt. Following overnight stirring, DCM was removed under reduced pressure, the residue was suspended in 50 mL of saturated NaHCO₃ solution and extracted with ethyl acetate (EtOAc, 4 x 50 mL). Combined organic layers were then washed with 5% LiCl solution (2 × 100 mL), dried over anhydrous Na₂SO₄, filtered, and concentrated in vacuo to give 2-bromo-N-(1-hydroxy-2-methylpropan-2-yl)benzamide as a crude brown oil, used in the next step without further purification.

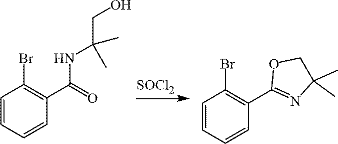

2-bromophenyl)-4,4-dimethyl-4,5-dihydrooxazole: 2-bromo-N-(1-hydroxy-2-methylpropan-2-yl) benzamide (ca. 6.5 g, 23.9 mmol, 1.0 equiv.) was transferred to an oven-dried round-bottom flask with DCM and purged with N₂. The crude oil was then treated with 6 molar equivalents of SOCl₂ (10.4 mL, 398.37 mmol, 6 equiv.) and stirred at rt for 1 hour. SOCl₂ was then removed under reduced pressure, and the residue was neutralized by the careful addition of 50 mL saturated NaHCO₃ solution in an ice bath. The aqueous layer was extracted with DCM (3 × 50 mL), and the organic layers were combined, dried over anhydrous Na₂SO₄, filtered, and concentrated in vacuo. The crude oil was purified by column chromatography with DCM containing 1% MeCN used as eluent to afford 2-(2-bromophenyl)-4,4-dimethyl-4,5-dihydrooxazole (4.87 g, 87%) as an amber oil. HRMS(ESI) [M+H]⁺ m/z found 254.0196, calcd for C₁₁H₁₃BrNO 254.0175.

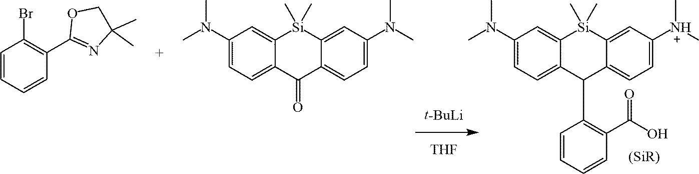

N-(2-carboxyphenyl)-7-(dimethylamino)-5,5-dimethyldibenzo[b,e]silin-3(5H)-ylidene)-N-methylmethanaminium (SiR): A flame-dried 100 mL round-bottom flask was charged with compound 2-(2-bromophenyl)-4,4-dimethyl-4,5-dihydrooxazole (0.79 g, 3.11 mmol, 2.0 equiv.), a magnetic stir bar and purged under N₂. Anhydrous THF (10 mL) was transferred to the flask via a syringe. The solution was then chilled to -78° C. in a bath of dry ice and acetone over 30 minutes prior to the addition of tert-butyllithium (t-BuLi, 1.7 M in hexanes, 2.01 mL, 3.42 mmol, 2.2 equiv.) dropwise over 10 minutes. The resultant mixture was stirred for 1 hour at -78° C. before a solution of compound 3,7-bis(dimethylamino)-5,5-dimethyldibenzo[b,e]silin-10(5H)-one (Koide et al., ACS Chemical Biology 6, 600-608 (2011))(0.51 g, 1.56 mmol, 1.0 equiv.) in anhydrous THF (10 mL) was added dropwise. The mixture was then removed from the cold bath and stirred for 3.5 hours under ambient conditions. Upon cooling in ice water bath, the mixture was carefully quenched with addition of 25 mL saturated NaHCO₃ solution, and the aqueous layer was extracted with DCM (4 × 50 mL). The combine organic layers were dried over anhydrous Na₂SO₄, filtered, and concentrated in vacuo. Purification of title compound (SiR) by column chromatography with methanol (MeOH) and DCM mixture as eluent afforded title compound (172 mg, 26%) as a light blue solid. HRMS(ESI) [M+H]⁺ m/z found 429.2026, calcd for C₂₆H₂₉N₂O₂Si 429.1993.

### 2-Bromo(Dimethylamino)Benzaldehyde (12)

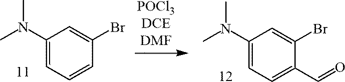

Synthesis 1) Compound 12 was synthesized by a protocol slightly modified from that published by Hanaoka et al.²⁰ In an oven-dried flask, under N₂ anhydrous DMF (8.51 mL, 110 mmol) was dissolved in 20 mL dichloroethane (DCE). The resultant mixture was chilled to ca. 0° C. prior to addition of POCl₃ (4.70 mL, 50.0 mmol), then stirred at 0° C. for 15 minutes. To the mixture was added a solution of compound 11 (5.00 g, 25.0 mmol) in anhy. DCE (20 mL) before the reaction was placed on an oil bath, heated to ca. 85° C. and allowed to reflux for 3 hours. Once cooled to rt, the reaction vessel was chilled to 0° C., neutralized by addition of 1 M NaOH solution, and the aqueous layer extracted with EtOAc (4× 50 mL). The combined organic layers were then washed with a 5% LiCl (2× 50 mL) solution, dried over anhy. Na₂SO₄, filtered, and concentrated in vacuo to give a crude amber oil. Compound was purified by flash column chromatography with CHCl₃ as eluent to afford compound 12 (5.04 g, 88.3%) of an off-white solid. HRMS(ESI) [M+H]⁺ m/z found 228.0019, calcd for C₉H₁₁BrNO 228.0018.

Synthesis 2) 2-bromo-4-(dimethylamino)benzaldehyde (12): Compound 11 was synthesized by a protocol slightly modified from that published by Hanaoka and Urano.⁹ In an oven-dried flask, under N₂ anhydrous DMF (17 mL, 220 mmol, 4.4 equiv.) was dissolved in 20 mL anhydrous dichloroethane (DCE). The resultant mixture was chilled to ca. 0° C. prior to the addition of phosphorus(V) oxychloride (POCl₃, 9.4 mL, 100 mmol, 2.0 equiv.), then stirred at 0° C. for 15 minutes. To the mixture was added a solution of compound 11 (10.0 g, 50.0 mmol, 1.0 equiv.) in anhydrous DCE (20 mL) before the reaction was placed on an oil bath, heated to ca. 85° C. and allowed to reflux for 3 hours. Once cooled to rt, the reaction vessel was chilled to 0° C., neutralized by addition of 1 M NaOH solution, and the aqueous layer extracted with EtOAc (4 × 50 mL). The combined organic layers were then washed with a 5% LiCl (2 × 100 mL) solution, dried over anhydrous Na₂SO₄, filtered, and concentrated in vacuo to give a crude amber oil. Crude product was purified by column chromatography with CHCl₃ as eluent to afford compound 12 (10.1 g, 88.4%) of a pale yellow/off-white solid. HRMS(ESI) [M+H]⁺ m/z found 228.0019, calcd for C₉H₁₁BrNO 228.0018.

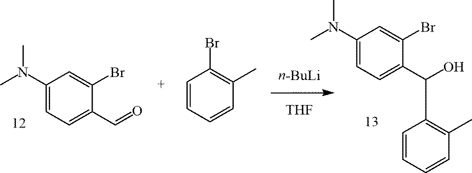

bromo-4-(dimethylamino)phenyl)(o-tolyl)methanol (13): A flame-dried 100 mL round-bottom flask was charged with a magnetic stir bar and purged under N₂ prior to the addition of 1-bromo-2-methylbenzene (2.44 mL, 20.3 mmol, 1.2 equiv.) via a syringe. This was then suspended in 20 mL anhydrous THF and chilled to -78° C. in a bath of dry ice in acetone over 30 minutes before n-butyllithium (n-BuLi, 1.6 M in hexanes, 12.7 mL, 20.3 mmol, 1.2 equiv.) solution was added dropwise over ca. 20 minutes. The resultant mixture was stirred at -78° C. for 45 minutes before compound 12 (3.85 g, 16.9 mmol, 1.0 equiv) was added in one portion as a solution in anhydrous THF (20 mL) and stirred at -78° C. for one additional hour. The reaction was then removed from the cold bath and stirred at rt for 1 hour. Upon cooling in ice water bath, the excess n-BuLi was quenched by the addition of 25 mL water and the aqueous layer was extracted with diethyl ether (Et₂O, 1 × 50 mL), followed by DCM (3 × 50 mL). Combined organic layers were dried over anhydrous Na₂SO₄, filtered, and concentrated in vacuo to give a crude, light yellow solid. Purification by column chromatography with a gradient (1-15%) of MeCN in DCM as eluent to afford a clear oil (4.4 g, 81%). HRMS(ESI) [M+H]⁺ m/z found 320.0635, calcd for C₁₆H₁₉BrNO 320.0645.

### N-AllylBromo-4-((2-Bromo-4-(Dimethylamino)Phenyl)(O-Tolyl)Methyl)-N-Methylaniline (14)

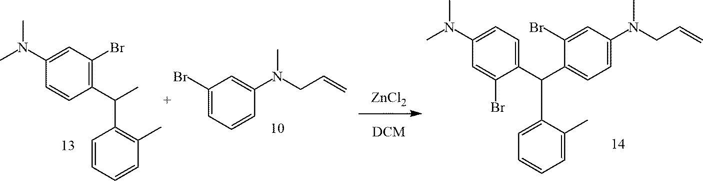

A flame-dried 100 mL round-bottom flask was charged with a magnetic stir bar and purged under N₂ for ca. 30 minutes. Compounds 10 (4.00 g, 12.49 mmol) and 13 (2.82 g, 12.49 mmol) were suspended with anhydrous DCM (40 mL). To the mixture was added ZnCl₂ (10.2 g, 75.0 mmol) rapidly, in one portion. The flask was placed on an oil bath, heated to 40° C., and refluxed for 4 hours. The reaction mixture was then cooled to rt, chilled to 0° C. and quenched by the careful addition of 50 mL saturated NaHCO₃ solution. The aqueous layer was extracted with DCM (3× 50 mL) and the organic layers were combined, dried over anhydrous Na₂SO₄, filtered, and concentrated in vacuo. The resulting crude product was purified by flash column chromatography with ethyl acetate/hexanes (1:5) + acetonitrile (1% v/v) to afford a pale-yellow solid (3.68 g, 56%). HRMS(ESI) [M+H]⁺ m/z found 529.0690, calcd for C₂₆H₂₉Br₂N₂ 529.0672.

### N³-Allyl-N³,N⁷,N⁷,5,5-Pentamethyl-10-(O-Tolyl)-5,10-Dihydrodibenzo[b,e]Siline-3,7-Diamine (15)

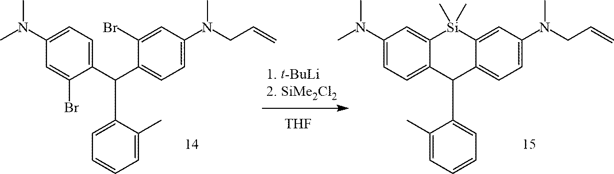

Compound 15 was synthesized according to a protocol modified slightly from that published by Bachman, et al.¹⁰ A flame-dried 100 mL round-bottom flask was charged with 14 (3.68 g, 6.96 mmol, 1.0 equiv.), a magnetic stir and purged under N₂ before bar before anhydrous tetrahydrofuran (THF, 20 mL) was added via a syringe. This was then chilled to -78° C. in a bath of dry ice and acetone over 30 minutes prior to the addition of t-BuLi (1.7 M in hexanes, 12.3 mL, 20.9 mmol, 3.0 equiv.) solution dropwise over 20 minutes. The resultant mixture was stirred for 30 minutes at -78° C. before dichlorodimethylsilane (1.51 mL, 12.5 mmol, 1.8 equiv.) was added in two portions, approximately 5 minutes apart. The mixture was stirred at -78° C. for an additional 10 minutes, then removed from the cold bath and stirred for 2 hours at ambient temperature. Upon cooling in ice water bath, excess t-BuLi was destroyed via careful addition of 25 mL saturated NaHCO₃ solution, and the aqueous layer was extracted with DCM (4 × 50 mL). The combined organic layers were dried over anhydrous Na₂SO₄, filtered, and concentrated in vacuo. The crude product was loaded directly onto an automatic flash purification system using silica gel with a mobile phase of EtOAc/Hexanes (gradient, 10-25% of EtOAc in Hexanes), afforded the product (1.78 g, 80%) as a light blue solid. HRMS(ESI) [M+H]⁺ m/z found 427.2545, calcd for C₂₈H₃₅N₂Si 427.2564.

### N³,N³,N⁷,5,5-Pentamethyl-10-(O-Tolyl)-5,10-Dihydrodibenzo[b,e]Siline-3,7-Diamine (16)

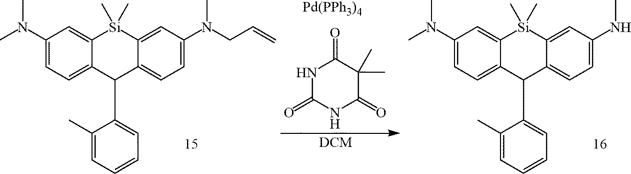

A flame-dried 100 mL round-bottom flask was charged with a magnetic stir bar and purged under N₂. Tetrakis(triphenylphosphine)palladium(0) (201 mg, 174.23 µmol, 0.13 equiv.) and dimethylbarbituric acid (DMBA,984 mg, 6.30 mmol, 4.7 equiv.) were added quickly. A solution of 29 (702 mg, 1.34 mmol, 1.0 equiv.) in anhydrous DCM (15 mL) was added via syringe and the resultant mixture was placed on an oil bath, heated to ca. 35° C., and stirred for 16 hours. The reaction was cooled to ambient temperature and DCM was removed under reduced pressure to afford a crude, dark green residue that was suspended in 15 mL MeOH, chilled in an ice bath, and treated with a minimal amount of NaBH₄ (ca. 5.0 equiv.) until the green color was no longer visible. Solvent was again removed under reduced pressure, suspended in a saturated solution of NaHCO₃ and extracted with DCM (3 × 25 mL). The combined organic layers were then dried over anhydrous Na₂SO₄, filtered, and concentrated in vacuo. The crude product was loaded directly onto an automatic flash purification system using silica gel with a mobile phase of EtOAc/Hexanes (gradient, 10-25% of EtOAc in Hexanes), afforded the product (354 mg, 68%) as a light blue solid. HRMS(ESI) [M+H]⁺ m/z found 387.2271, calcd for C₂ₛH₃₁N₂Si 387.2251.

### E)((7-(Dimethylamino)-5,5-Dimethyl-10-(O-Tolyl)Dibenzo[b,e]Silin-3(5H)-Ylidene)(Methyl)Ammonio)Propane-1-Sulfonate (Sulfo-SiRh, OF650)

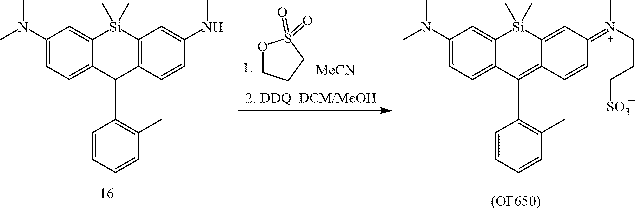

A 100 mL round-bottom flask was charged with compound 16 (250 mg, 645 µmol, 1.0 equiv.) and a magnetic stir bar, then purged under N₂. The compound was suspended in 35 mL anhydrous MeCN before 1,3-propanesultone (68 µL, 774 µmol, 1.2 equiv.) was added in a single portion. The reaction was placed on an oil bath, heated to 83° C., and refluxed for ca. 24 h. Afterwards, the reaction was cooled to rt prior to addition of an additional 2.5 equivalents sultone. The reaction was then stirred at reflux for an additional 24 h, before cooling to rt, diluted with 50 mL DCM, and filtered through a pre-packed Celite funnel. Organic solvents were removed under reduced pressure to give the intermediate product as a white solid. In a 100 mL round-bottom flask, this intermediate was suspended in 20 mL DCM/MeOH (3:1) and chilled in an ice bath for 15 minutes. DDQ (220 mg, 967 µmol, 1.5 equiv.) was then slowly added to the mixture. This was allowed to stir at 0° C. for another 15 minutes before solvent was removed under reduced pressure to give a crude, highly viscous blue resin. The crude product was loaded directly onto an automatic flash purification system using silica gel with a mobile phase of DCM/MeOH containing 0.5% formic acid (gradient, 2-15% of MeOH in DCM) to give OF650 (178 mg, 54%) as a blue solid. HRMS(ESI) [M+H]⁺ m/z found 507.2170, calcd for C₂₈H₃₅N₂O₃SSi 507.2132.

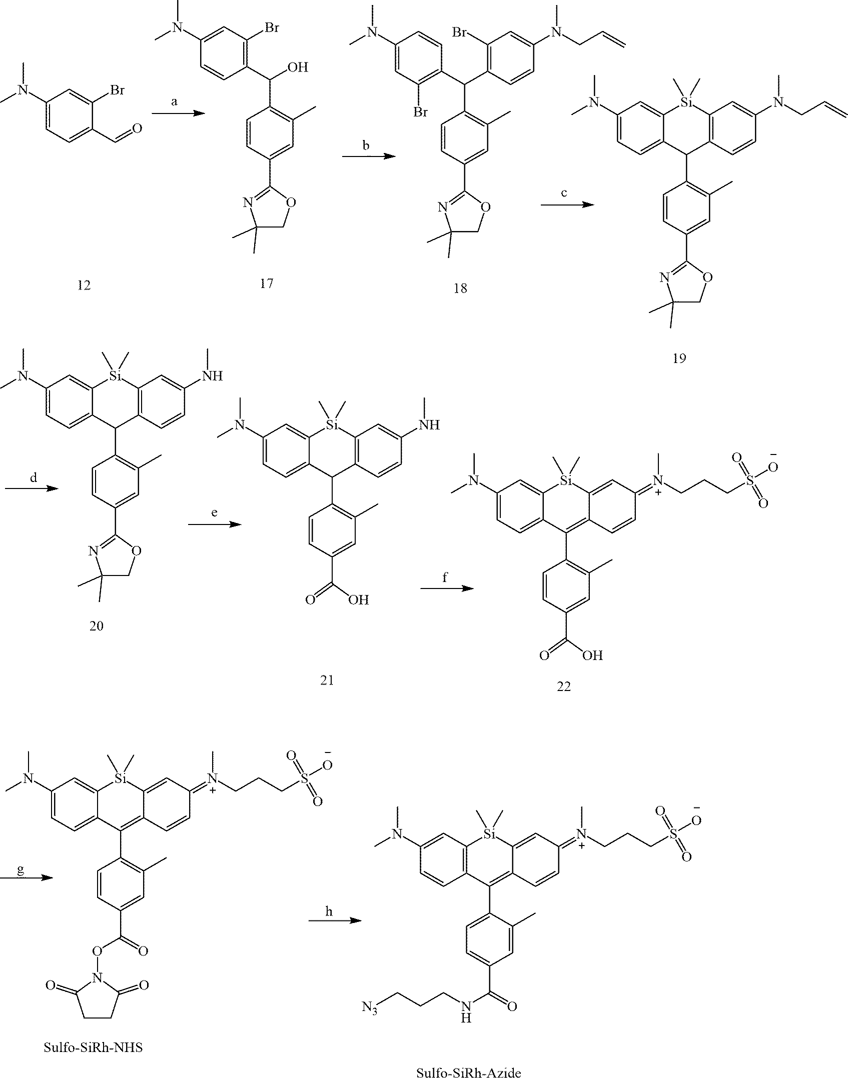

Scheme 4 - Synthetic route to Sulfo-SiRh-NHS and Sulfo-SiRh-Azide: Reagents and conditions: a) i) Compound 7, n-BuLi, -78° C. to rt; b) i) Compound 10, ZnCl₂, DCM, 34° C.; c) i) t-BuLi, THF, -78° C.; ii) dichlorodimethylsilane, -78° C. to rt; d) i) Pd(PPh₃)₄, dimethylbarbituric acid (DMBA), DCM, 35° C.; e) 6 M HCl, 80° C.; f) i) 1,3-propanesultone, MeCN, 85° C.; ii) DDQ,DCM, 0° C. g) N,N,N′,N′-tetramethyl-O-(N-succinimidyl)uronium tetrafluoroborate (TSTU), DiPEA, DMF, rt; h) 3-azido-1-propanamine, DiPEA, DMF, rt.

### Bromo-4-(Dimethylamino)Phenyl)(4-(4,4-Dimethyl-4,5-Dihydrooxazol-2-yl)-2-Methylphenyl)Methanol (17)

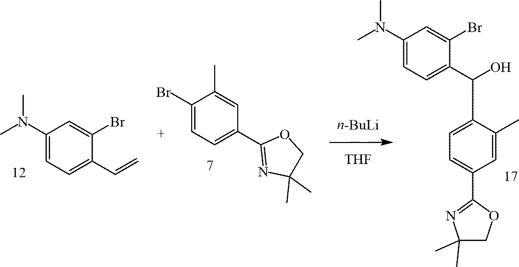

A flame-dried 100 mL round-bottom flask was charged with a magnetic stir bar and purged under N₂ for ca. 30 minutes prior to the addition of 2-(4-bromo-3-methylphenyl)-4,4-dimethyl-4,5-dihydrooxazole 7 (8.81 g, 32.9 mmol) as a solution in anhy. DCM (20 mL). The solution was then chilled to ca. -78° C. in a bath of dry ice in acetone over 30 minutes under N₂ before n-BuLi (1.6 M in hexanes, 24.7 mL, 39.4 mmol) was added dropwise over ca. 20 minutes. The reaction was then stirred at -78° C. for 45 minutes before 3-bromo-N,N-dimethyl-4-vinylaniline 12 (8.99 g, 39.44 mmol) suspended in anhy. DCM (20 mL) was added in one portion. The resulting mixture was then stirred at -78° C. for one additional hour. Afterwards, the reaction was removed from the cold bath and stirred at rt for 1 hour before excess BuLi was quenched by the addition of 25 mL DI water. The aqueous layer was first extracted with Et₂O (1× 50 mL), followed by DCM (3× 50 mL). The combined organic layers were dried over anhy. Na₂SO₄, filtered, and concentrated in vacuo to give a crude, light yellow solid. Purification by column chromatography with a gradient (1-15%) of MeCN in DCM as eluent to afford an off-white solid (10.1 g, 74%). [M+H]⁺ m/z found 417.1052, calcd for C₂₁H₂₆BrN₂O₂ 417.1172.

### N-AllylBromo-4-((2-Bromo-4-(Dimethylamino)Phenyl)(4-(4,4-Dimethyl-4,5-Dihydrooxazol-2-yl)-2-Methylphenyl)methyl)-N-Methylaniline (18)

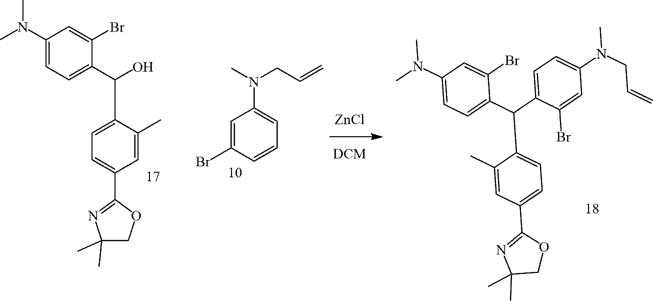

A flame-dried 100 mL round-bottom flask was charged with a magnetic stir bar and purged under N₂ for ca. 30 minutes. (2-bromo-4-(dimethylamino)phenyl)(4-(4,4-dimethyl-4,5-dihydrooxazol-2-yl)-2-methylphenyl)methanol 17 (2.67 g, 6.41 mmol) and N-allyl-3-bromo-N-methylaniline 10 (1.59 g, 7.05 mmol) were suspended in anhydrous DCM (40 mL). To the mixture was added ZnCl₂ (5.24 g, 38.5 mmol), rapidly, in one portion. The flask was placed on an oil bath, heated to 35° C., and refluxed for 4 hours. The reaction mixture was then chilled to 0° C. and quenched by the careful addition of 50 mL saturated NaHCO₃ solution. The aqueous layer was extracted with DCM (3× 50 mL) and the organic layers were combined, dried over anhy. Na₂SO₄, filtered, and concentrated in vacuo. Compound 18 was purified by column chromatography with EtOAc/hexanes (1:5) containing MeCN (1% v/v) to afford a pale-yellow solid (2.65 g, 66%). HRMS(ESI) [M+H]⁺ m/z found 626.1247, calcd for C₃₁H₃₆Br₂N₃O 626.1200.

### N³-Allyl-10-(4-(4,4-Dimethyl-4,5-Dihydrooxazol-2-yl)-2-Methylphenyl)-N³,N⁷,N⁷,5,5-Pentamethyl-5,10-Dihydrodibenzo[b,e]Siline-3,7-Diamine (19)

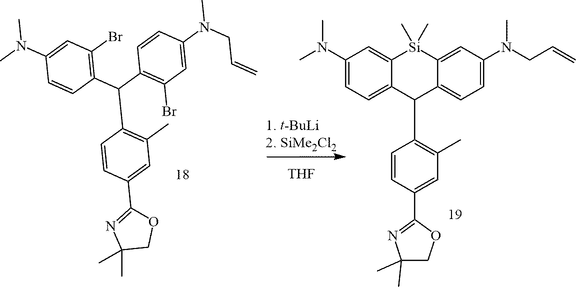

A flame-dried 100 mL round-bottom flask was charged with a magnetic stir bar and purged under N₂ for ca. 30 minutes before a solution of compound 18 (2.65 g, 4.24 mmol) in THF (40 mL) was transferred via syringe. The solution was then chilled to -78° C. in a bath of dry ice and acetone over 30 minutes prior to the addition of t-BuLi (1.7 M in hexanes, 9.08 mL, 12.7 mmol) dropwise over 15 minutes. The resultant mixture was stirred for 30 minutes at -78° C. before dichlorodimethylsilane (0.92 mL, 7.63 mmol) was added in two portions, ca. 5 minutes apart. The mixture was stirred at -78° C. for an additional 10 minutes, then removed from the cold bath and stirred for 2 hours under ambient conditions. Excess BuLi was destroyed via careful addition of 25 mL saturated NaHCO₃ solution, and the aqueous layer was extracted with DCM (4× 50 mL). The combined organic layers were dried over anhydrous Na₂SO₄, filtered, and concentrated in vacuo to give a viscous dark green oil. Purification of 19 by column chromatography with EtOAc/hexanes (¼) as eluent afforded a highly viscous, bright green oil (1.78 g, 80%). HRMS(ESI) [M+H]⁺ m/z found 524.3069, calcd for C₃₃H₄₂N₃OSi 524.3092.

### 10-(4,4-Dimethyl-4,5-Dihydrooxazol-2-yl)-2-Methylphenyl)-N³,N³,N⁷,5,5-Pentamethyl-5,10-Dihydrodibenzo[b,e]Siline-3,7-Diamine (20)

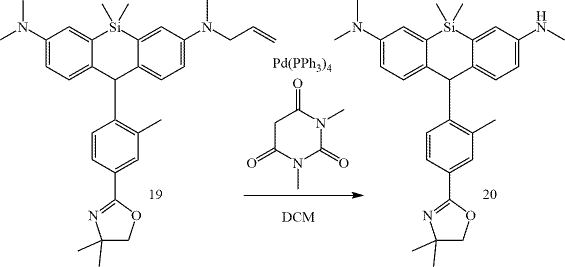

A flame-dried 100 mL round-bottom flask was charged with a magnetic stir bar and purged under N₂ for ca. 30 minutes. Pd(PPh₃)₄ (238 mg, 0.442 mmol) and dimethylbarbituric acid (2.49 g, 16.0 mmol) were added in a single portion under N₂. A solution of 19 (1.78 g, 3.40 mmol) in anhydrous DCM (20 mL) was then added via syringe and the resultant mixture was placed on an oil bath, heated to ca. 35° C., and stirred for 16 hours. The reaction was cooled to ambient temperature and DCM removed under reduced pressure to afford a crude, dark green residue that was re-suspended in 15 mL methanol, chilled in ice, and treated with a minimal amount of NaBH₄ until the green color was no longer visible. Solvent was again removed under reduced pressure, suspended in a saturated solution of NaHCO₃ and extracted with DCM (3× 50 mL). The combined organic layers were then dried over anhydrous Na₂SO₄, filtered, and concentrated in vacuo. Compound was purified by column chromatography to give 20 as a highly viscous blue resin (1.54 g, 94%). HRMS(ESI) [M+H]⁺ m/z found 484.2804, calcd for C₃₀H₃₈N₃OSi 484.2779.

### 4-(Dimethylamino)-5,5-Dimethyl-7-(Methylamino)-5,10-Dihydrodibenzo[b,e]Silin-10-yl)-3-Methylbenzoate (21)

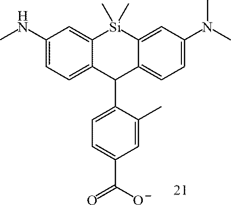

A 250 mL round-bottom flask was charged with compound 20 (1.54 g, 3.18 mmol) and a magnetic stir bar. To the flask was added 6 M HCl (16 mL, 28 equiv.). The resultant mixture was then placed on an oil bath and heated to 80° C. for 24 h. The reaction was then cooled to rt and the pH of the solution was adjusted to 2-4 with 50% NaOH solution. The aqueous layer was extracted with DCM (3× 25 mL) and EtOAc (2× 25 mL). The combined organic layers were dried over anhydrous Na₂SO₄, filtered, and concentrated in vacuo. The residue was then suspended in 15 mL MeOH and treated with a minimal amount of NaBH₄ until the blue color was no longer visible. The MeOH was then removed under reduced pressure and the crude product was loaded directly onto a Biotage Isolera Flash System using a SNAP Ultra cartridge with a mobile phase of EtOAc/Hexanes (2:3) to give the 21 as a viscous blue liquid (630.3 mg, 46%). HRMS(ESI) [M+H]⁺ m/z found 431.2193, calcd for C₂₆H₃₁N₂O₂Si 431.2149.

### E)(7-(dimethylamino)-5,5-dimethyl-3-(methyl(3-sulfonatopropyl)iminio)-3,5-dihydrodibenzo[b,e]silin-10-yl)-3-methylbenzoate (22)

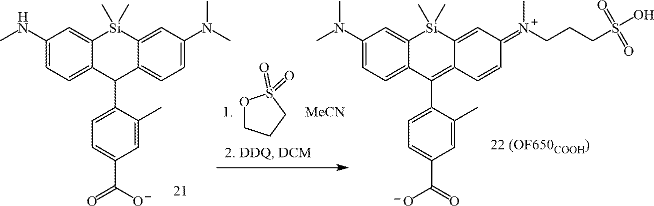

Synthesis 1) A 100 mL round-bottom flask was charged with compound 21 (630 mg, 1.46 mmol) and a magnetic stir bar, then purged for 20 minutes under N₂. The compound was then suspended in 20 mL anhydrous MeCN before 1,3-propanesultone (268 mg, 2.20 mmol) was added in a single portion. The reaction was placed on an oil bath, heated to 83° C., and refluxed for ca. 24 h. Afterwards, the reaction was cooled to rt prior to addition of an additional 1.5 equivalents sultone. The reaction was then stirred at reflux for an additional 24 h, before cooling to rt, diluting with 50 mL DCM, and filtering through a pre-packed Celite funnel. Organic solvent was removed under reduced pressure to give the product as a highly viscous blue oil. The residue was purified on a Biotage Isolera Flash System using a SNAP Ultra cartridge with a mobile phase of DCM and MeOH containing 5% MeCN and 1% formic acid (gradient, 5-20% of MeOH in DCM). The fractions containing the product were pooled and contracted in vacuo. Final purification was completed on a reverse phase prep-HPLC system.

Synthesis 2) A 100 mL round-bottom flask was charged with compound 21 (100 mg, 232 µmol, 1.0 equiv.) and a magnetic stir bar, then purged under N₂. The compound was then suspended in 5 mL anhydrous MeCN before 1,3-propanesultone (222 µL, 348 µmol, 1.5 equiv.) was added in a single portion. The reaction was placed on an oil bath, heated to 83° C., and refluxed for ca. 24 h. Upon cooling to rt, the reaction mixture was diluted with t mL DCM, and filtered through a pre-packed Celite funnel. Organic solvents were removed under reduced pressure. In a 100 mL round-bottom flask, this intermediate was suspended in 5 mL DCM/MeOH (3:1) and chilled in an ice bath for 15 minutes. DDQ (80 mg, 348 µmol, 1.5 equiv.) was then slowly added to the mixture. This was allowed to stir at 0° C. for another 15 minutes before solvent was removed under reduced pressure to give a crude, highly viscous blue resin. The crude product was loaded directly onto an automatic flash purification system using silica gel with a mobile phase of DCM/MeOH containing 0.5% formic acid (gradient, 5-20% of MeOH in DCM). The fractions containing the product were pooled and contracted in vacuo. Final purification was completed on a reverse phase prep-HPLC system. The title compound (42 mg, 33%) was obtained as a blue solid. HRMS(ESI) [M+H]⁺ m/z found 551.2070, calcd for C₂₉H₃₅N₂O₅SSi 551.2030. Compound 22 may also be named (E)-3-((10-(4-carboxy-2-methylphenyl)-7-(dimethylamino)-5,5-dimethyldibenzo[b,e]silin-3(5H)-ylidene)(methyl)ammonio)propane-1-sulfonate.

### E)((7-(Dimethylamino)-10-(4-(((2,5-Dioxopyrrolidin-1-yl)Oxy)Carbonyl)-2-Methylphenyl)-5,5-Dimethyldibenzo[b,e]Silin-3(5H)-Ylidene)(Methyl)Ammonio)Propane-1-Sulfonate (Sulfo-SiRh-NHS)

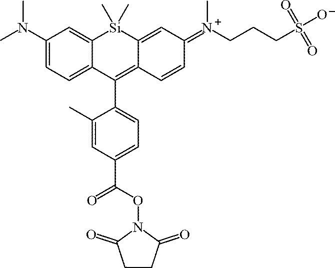 [SMILES: Cc1cc(C(=O)ON2C(=O)CCC2=O)ccc1C1=C2C=C/C(=[N+](/C)CCCS(=O)(=O)[O-])C=C2[Si](C)(C)c2cc(N(C)C)ccc21]

Under N₂, a scintillation vial was charged with compound 22 (60 mg, 0.109 mmol) and TSTU (66 mmg, 0.218 mmol). The vial was purged under N₂ for an additional 15 min before adding 1 mL anhydrous DMF, followed by DiPEA (57 µL, 0.327 mmol). The reaction mixture was covered from light and stirred at rt for 2 h. The crude product was purified by reverse phase prep-HPLC (10-90% MeCN/H₂O gradient with 0.1% TFA), afforded the title compound (49 mg, 69%) as a dark blue solid. HRMS(ESI) [M+H]⁺ m/z found 648.2258, calcd for C₃₃H₃₈N₃O₇SSi 648.2194.

### E)((10-(4-((3-Azidopropyl)Carbamoyl)-2-Methylphenyl)-7-(Dimethylamino)-5,5-Dimethyldibenzo[b,e]Silin-3(5H)-Ylidene)(Methyl)Ammonio)Propane-1-Sulfonate (Sulfo-SiRh-Azide)

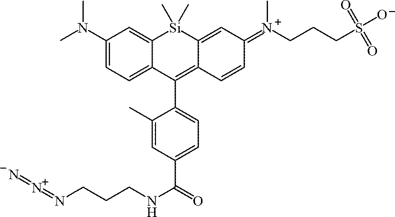 [SMILES: Cc1cc(C(=O)NCCCN=[N+]=[N-])ccc1C1=C2C=C/C(=[N+](/C)CCCS(=O)(=O)[O-])C=C2[Si](C)(C)c2cc(N(C)C)ccc21]

Under N₂, a scintillation vial was charged with compound Sulfo-SiRh-NHS (20 mg, 0.031 mmol) and 3-azido-1-propanamine (6.18 mg, 0.062 mmol). The vial was purged under N₂ for an additional 15 min before adding 0.5 mL anhydrous DMF, followed by DiPEA (16 µL, 0.093 mmol). The reaction mixture was covered from light and stirred at rt for 12 h. The crude product was purified by reverse phase prep-HPLC (10-90% MeCN/H₂O gradient with 0.1% TFA), afforded the title compound (15 mg, 77%) as a dark blue solid. HRMS(ESI) [M+H]⁺ m/z found 633.2658, calcd for C₃₂H₄₁N₆O₄SSi 633.2674.

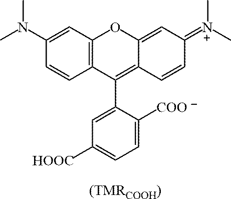

4-carboxy-2-(6-(dimethylamino)-3-(dimethyliminio)-3H-xanthen-9-yl)benzoate (TMR_{COOH}): TMR_{COOH} was prepared following previously reported protocol. Kvach et al., Bioconjugate Chemistry 20, 1673-1682 (2009).

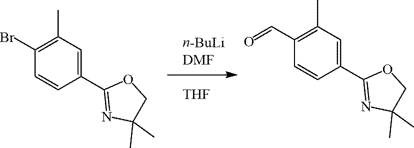

4-(4,4-dimethyl-4,5-dihydrooxazol-2-yl)-2-methylbenzaldehyde: A flame-dried 100 mL round-bottom flask was charged with a magnetic stir bar and purged under N₂ prior to the addition of 2-(4-bromo-3-methylphenyl)-4,4-dimethyl-4,5-dihydrooxazole (2.0 g, 7.46 mmol, 1.0 equiv.) as a solution in anhydrous THF (20 mL). The solution was then chilled to ca. -78° C. in a bath of dry ice in acetone over 30 minutes before n-BuLi (1.6 M in hexanes, 5.59 mL, 8.95 mmo, 1.2 equiv.) solution was added dropwise over ca. 20 minutes. The resultant mixture was then stirred at -78° C. for 45 minutes before DMF (0.81 mL, 10.4 mmol, 1.4 equiv.) was added in one portion and the mixture stirred for an additional hour at -78° C. The reaction was then removed from the cold bath and stirred at rt for 1 hour before excess BuLi was quenched by the addition of 25 mL water. The aqueous layer was then extracted with Et₂O (1 × 50 mL), followed by DCM (3 × 50 mL). Combined organic layers were dried over anhydrous Na₂SO₄, filtered, and concentrated in vacuo. Purification by column chromatography using silica gel with EtOAc/Hexanes (⅕) as eluent to give the product 4-(4,4-dimethyl-4,5-dihydrooxazol-2-yl)-2-methylbenzaldehyde as a clear oil (1.32 g, 81%). HRMS(ESI) [M+H]⁺ m/z found 218.1127, calcd for C₁₃H₁₆NO₂ 218.1176.

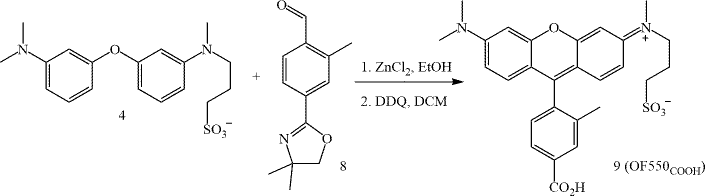

(E)((9-(4-carboxy-2-methylphenyl)-6-(dimethylamino)-3H-xanthenylidene)(methyl)ammonio)propane-1-sulfonate (OF550_{COOH}): A flame-dried 100 mL round-bottom flask was charged with a magnetic stir bar and purged under N₂. 3-((3-(3-(dimethylamino)phenoxy)phenyl)(methyl)amino)propane-1-sulfonate (348 mg, 920 µmol, 1.0 equiv.) and 4-(4,4-dimethyl-4,5-dihydrooxazol-2-yl)-2-methylbenzaldehyde (200 mg, 920 µmol, 1.0 equiv.) were combined and suspended in anhydrous EtOH (20 mL). To the mixture was added anhydrous ZnCl₂ (1.88 g, 13.8 mmol, 15 equiv.) rapidly, in one portion. The flask was placed on an oil bath, heated to 110° C., and refluxed for 8 hours. The solvent was removed under reduced pressure, and the residue was then resuspended in 6 M HCl (10 mL). The resulting mixture was heated at 78° C. and stirred overnight. The reaction was then removed from the oil bath and cooled to rt, then deposited onto a C18 cartridge, which was eluted with DI water followed by MeCN. The solution was concentrated in vacuo and the residue was suspended in 20 mL DCM/MeOH (3:1). The resulting solution was then chilled in an ice bath for 15 minutes before DDQ (313 mg, 1.38 mmol, 1.5 equiv.) was slowly added to the mixture. This was allowed to stir at 0° C. for another 15 minutes before organic solvent was removed under reduced pressure. The residue was purified on an automatic flash purification system with a mobile phase of DCM and MeOH containing 5% MeCN and 1% formic acid (gradient, 5-30% of MeOH in DCM). The fractions containing the product were pooled and contracted in vacuo. Final purification was completed on a prep-HPLC system. The title compound (113 mg, 24 %) was obtained as a dark red solid. HRMS(ESI) [M+H]⁺ m/z found 509.1718, calcd for C₂₇H₂₉N₂O₆S 509.1741.

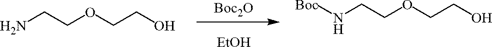

tert-butyl (2-(2-hydroxyethoxy)ethyl)carbamate: Under N₂, 2-(2-aminoethoxy)ethan-1-ol (9.5 mL, 95.1 mmol, 1.0 equiv.) was suspended in anhydrous EtOH (100 mL). The reaction solution was chilled in an ice bath for ca. 30 minutes before Boc₂O (20.8 g, 97.0 mmol, 1.02 equiv.) was added in three portions over 30 minutes. The reaction mixture was slowly warmed up to rt and stirred overnight. The reaction solvent was then removed under reduced pressure. The residue was redissolved in DCM (50 mL), washed with water (50 mL), then brine (50 mL), subsequently dried over anhydrous Na₂SO₄, filtered, and concentrated in vacuo. The crude intermediate tert-butyl (2-(2-hydroxyethoxy)ethyl)carbamate was used in the next step without further purification.

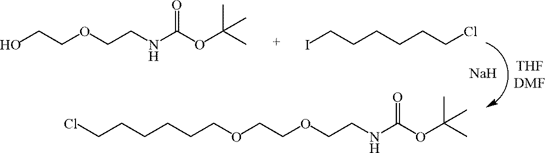

tert-butyl (2-(2-((6-chlorohexyl)oxy)ethoxy)ethyl)carbamate: A flame-dried 250 mL round-bottom flask was charged with a magnetic stir bar and purged under N₂. Tert-butyl (2-(2-hydroxyethoxy)ethyl)carbamate (ca. 2 g, 9.74 mmol, 1.0 equiv.) was suspended in a mixture of anhydrous THF (10 mL) and anhydrous DMF (10 mL). The reaction solution was then chilled to in an ice water bath over 30 minutes before NaH (60%, 546 mg, 13.6 mmol, 1.4 equiv.) was added in 3 portions over ca. 15 minutes. The resultant mixture was stirred for 30 minutes at 0° C. before 1-chloro-6-iodohexane (2.27 mL, 14.6 mmol, 1.5 equiv.) was added. The mixture was stirred at 0° C. for an additional 30 minutes before removed from the ice bath, and then stirred under ambient conditions overnight. Reaction was monitored using TLC with ninhydrin stain. Upon cooling in ice water bath, the mixture was quenched with addition of 50 mL saturated NH₄Cl solution, and the aqueous layer was extracted with EtOAc (2 × 100 mL). The combined organic layers were washed with brine (50 mL), dried over anhydrous Na₂SO₄, filtered, and concentrated in vacuo. Purification by column chromatography with a gradient (20-35%) of EtOAc in Hexanes as eluent to afford a clear oil (1.57 g, 50%). HRMS(ESI) [M+Na]⁺ m/z found 346.1736, calcd for C₁₅H₃₀ClNNaO₄ 346.1756.

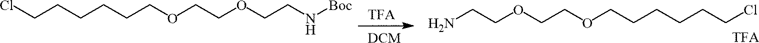

2-((6-chlorohexyl)oxy)ethoxy)ethan-1-amine: A 100 mL round bottom flask was charged with tert-butyl (2-(2-((6-chlorohexyl)oxy)ethoxy)ethyl)carbamate (850 mg, 2.62 mmol, 1.0 equiv.), a magnetic stir bar and DCM (20 mL). The reaction solution was chilled in an ice bath for ca. 20 minutes before TFA (3.45 mL) was slowly added. The reaction mixture was stirred in the ice bath for 2.5 hours before concentrated in vacuo. The crude product was purified by reverse phase prep-HPLC (10-90% MeCN/H₂O gradient with 0.1% TFA). The fractions containing the product verified by nihydrin stain and LCMS were pooled and lyophilized. The title compound, 2-(2-((6-chlorohexyl)oxy)ethoxy)ethan-1-amine (374 mg, 64%), was obtained as a viscos clear oil. HRMS(ESI) [M+H]⁺ m/z found 224.1405, calcd for C₁₀H₂₃ClNO₂ 224.1412.

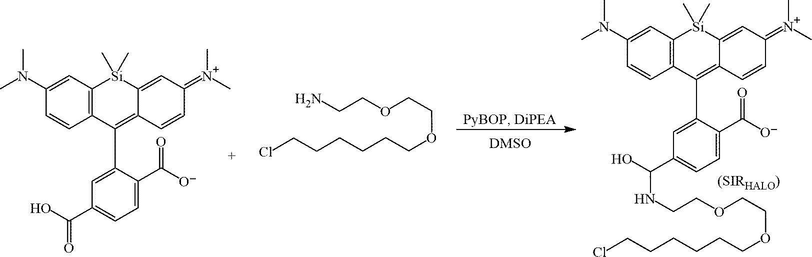

4-(((6-chlorohexyl)oxy)ethoxy)ethyl)carbamoyl)-2-(7-(dimethylamino)-3-(dimethyliminio)-5,5-dimethyl-3,5-dihydrodibenzo[b,e]silin-10-yl)benzoate (SiR_{HALO}): Under N₂, a 1 dram glass vial was charged with 4-carboxy-2-(7-(dimethylamino)-3-(dimethyliminio)-5,5-dimethyl-3,5-dihydrodibenzo [b,e]silin-10-yl)benzoate (12 mg, 25.3 µmol, 1.0 equiv.), 2-(2-((6-chlorohexyl)oxy) ethoxy)ethan-1-amine (17 mg, 50.1 µmol, 2.0 equiv.), and PyBOP (20 mg, 38 µmol, 1.5 equiv.). The vial was purged under N₂ for an additional 15 min before adding 0.5 mL anhydrous DMSO, followed by DiPEA (27 µL, 152 µmol, 6.0 equiv.). The reaction mixture was covered from light and stirred at rt for 3 hours. The crude product was purified by reverse phase prep-HPLC (10-90% MeCN/H₂O gradient with 0.1% TFA). The title compound (10.1 mg, 59%) was obtained as a dark blue solid. Structural characterization data for compound SiR_{HALO} matched that was previously published.¹² HRMS(ESI) [M+H]⁺ m/z found 678.3041, calcd for C₃₇H₄₉ClN₃O₅Si 678.3125.

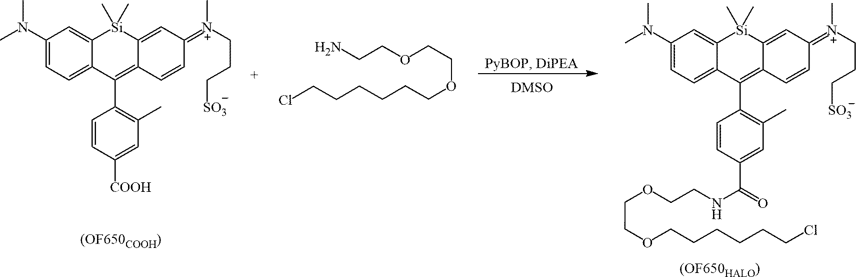

(E)((10-(4-((2-(2-((6-chlorohexyl)oxy)ethoxy)ethyl)carbamoyl)-2-methylphenyl)-7-(dimethylamino)-5,5-dimethyldibenzo[b,e]silin-3(5H)-ylidene)(methyl)ammonio)propane-1-sulfonate (OF650_{HALO}): Under N₂, a 1 dram glass vial was charged with (E)-3-((10-(5-carboxy-2-methylphenyl)-7-(dimethylamino)-5,5-dimethyldibenzo[b,e]silin-3(5H)-ylidene) (methyl)ammonio)propane-1-sulfonate (10 mg, 18.2 µmol, 1.0 equiv.), 2-(2-((6-chlorohexyl) oxy)ethoxy)ethan-1-amine (13 mg, 36.3 µmol, 2.0 equiv.), and PyBOP (14 mg, 27.2 µmol, 1.5 equiv.). The vial was purged under N₂ for an additional 15 min before adding 0.5 mL anhydrous DMSO, followed by DiPEA (9.5 µL, 54.5 µmol, 3.0 equiv.). The reaction mixture was covered from light and stirred at rt for 3 hours. The crude product was purified by reverse phase prep-HPLC (10-90% MeCN/H₂O gradient with 0.1% TFA). The title compound (10 mg, 73%) was obtained as a dark blue solid. HRMS(ESI) [M+H]⁺ m/z found 756.3204, calcd for C₃₉H₅₅ClN₃O₆SSi 756.3264.

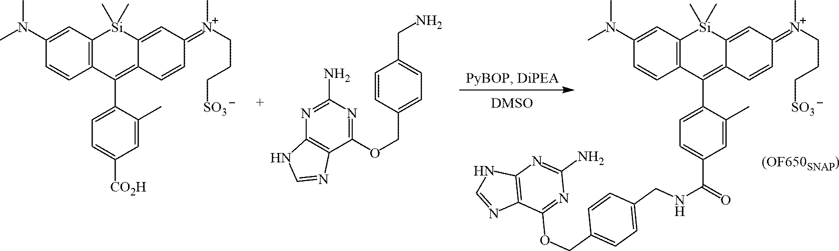

(E)((10-(4-((4-(((2-amino-9H-purin-6-yl)oxy)methyl)benzyl)carbamoyl)-2-methylphenyl)-7-(dimethylamino)-5,5-dimethyldibenzo[b,e]silin-3(5H)-ylidene)(methyl)ammonio)propane-1-sulfonate (11, OF650_{SNAP}): Under N₂, a 1 dram glass vial was charged with (E)-3-((10-(5-carboxy-2-methylphenyl)-7-(dimethylamino)-5,5-dimethyldibenzo[b,e]silin-3(5H)-ylidene) (methyl)ammonio) propane-1-sulfonate (10 mg, 18.2 µmol, 1.0 equiv.), 6-((4-(aminomethyl)benzyl)oxy)-9H-purin-2-amine (10 mg, 36.3 µmol, 2.0 equiv.), and PyBOP (14 mg, 27.2 µmol, 1.5 equiv.). The vial was purged under N₂ for an additional 15 min before adding 0.5 mL anhydrous DMSO, followed by DiPEA (9.5 µL, 54.5 µmol, 3.0 equiv.). The reaction mixture was covered from light and stirred at rt for 3 hours. The crude product was purified by reverse phase prep-HPLC (10-90% MeCN/H₂O gradient with 0.1% TFA). The title compound (8.7 mg, 60%) was obtained as a dark blue solid. HRMS(ESI) [M+H]⁺ m/z found 803.3120, calcd for C₄₂H₄₇N₈O₅SSi 803.3154.

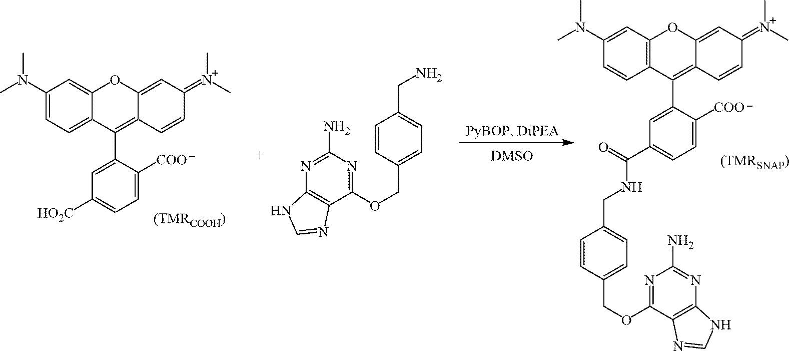

4-((((2-amino-9H-purin-6-yl)oxy)methyl)benzyl)carbamoyl)-2-(6-(dimethylamino)-3-(dimethyliminio)-3H-xanthen-9-yl)benzoate(12, TMR_{SNAP}): Under N₂, a 1 dram glass vial was charged with (E)-4-carboxy-2-(6-(dimethylamino)-3-(methyl(3-sulfonatopropyl)iminio)-3H-xanthen-9-yl)benzoate (10 mg, 23.2 µmol, 1.0 equiv.), 6-((4-(aminomethyl)benzyl)oxy)-9H-purin-2-amine (13 mg, 46.4 µmol, 2.0 equiv.), and PyBOP (18 mg, 34.8 µmol, 1.5 equiv.). The vial was purged under N₂ for an additional 15 min before adding 0.5 mL anhydrous DMSO, followed by DiPEA (24 µL, 139 µmol, 6.0 equiv.). The reaction mixture was covered from light and stirred at rt for 3 hours. The crude product was purified by reverse phase prep-HPLC (10-90% MeCN/H₂O gradient with 0.1% TFA). The title compound (10.5 mg, 66%) was obtained as a red solid. HRMS(ESI) [M+H]⁺ m/z found 683.2070, calcd for C₃₈H₃₅N₈O₅ 683.2725.

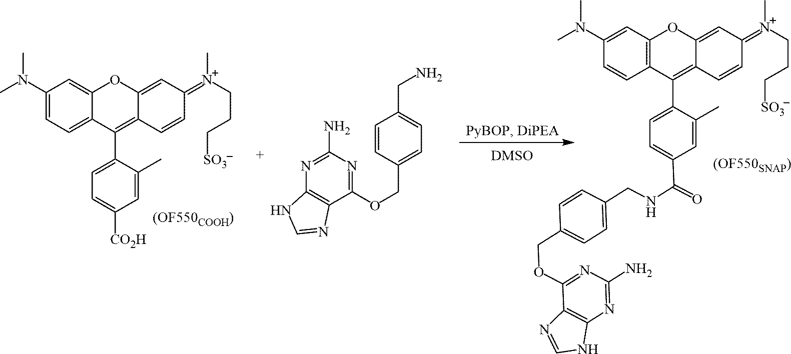

(E)((9-(4-((4-(((2-amino-9H-purin-6-yl)oxy)methyl)benzyl)carbamoyl)-2-methylphenyl)-6-(dimethylamino)-3H-xanthenylidene)(methyl)ammonio)propane-1-sulfonate (OF550_{SNAP}): Under N₂, a 1 dram glass vial was charged with (E)-3-((9-(4-carboxy-2-methylphenyl)-6-(dimethylamino)-3H-xanthen-3-ylidene)(methyl)ammonio)propane-1-sulfonate (10 mg, 23.2 µmol, 1.0 equiv.), 6-((4-(aminomethyl)benzyl)oxy)-9H-purin-2-amine (10 mg, 19.7 µmol, 2.0 equiv.), and PyBOP (16 mg, 29.5 µmol, 1.5 equiv.). The vial was purged under N₂ for an additional 15 min before adding 0.5 mL anhydrous DMSO, followed by DiPEA (11 µL, 60 µmol, 3.0 equiv.). The reaction mixture was covered from light and stirred at rt for 3 hours. The crude product was purified by reverse phase prep-HPLC (10-90% MeCN/H₂O gradient with 0.1% TFA). The title compound (9.7 mg, 65%) was obtained as a red solid. HRMS(ESI) [M+H]⁺ m/z found 761.2815, calcd for C₄₀H₄₁N₈O₆S 761.2864.

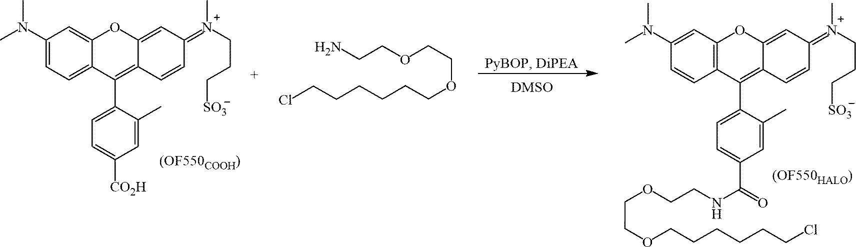

(E)((9-(4-((2-(2-((6-chlorohexyl)oxy)ethoxy)ethyl)carbamoyl)-2-methylphenyl)-6-(dimethylamino)-3H-xanthenylidene)(methyl)ammonio)propane-1-sulfonate (OF550_{HALO}): Under N₂, a 1 dram glass vial was charged with (E)-3-((9-(4-carboxy-2-methylphenyl)-6-(dimethylamino)-3H-xanthen-3-ylidene)(methyl)ammonio)propane-1-sulfonate (12 mg, 23.6 µmol, 1.0 equiv.), 2-(2-((6-chlorohexyl)oxy)ethoxy)ethan-1-amine (16 mg, 47.2 µmol, 2.0 equiv.), and PyBOP (19 mg, 35.4 µmol, 1.5 equiv.). The vial was purged under N₂ for an additional 15 min before adding 0.5 mL anhydrous DMSO, followed by DiPEA (12.3 µL, 70.8 µmol, 3.0 equiv.). The reaction mixture was covered from light and stirred at rt for 3 hours. The crude product was purified by reverse phase prep-HPLC (10-90% MeCN/H₂O gradient with 0.1% TFA). The title compound (11 mg, 65%) was obtained as a dark red solid. HRMS(ESI) [M+H]⁺ m/z found 714.2959, calcd for C₃₇H₄₉ClN₃O₇S 714.2974.

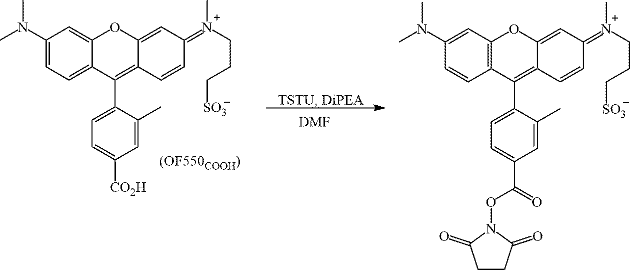

(E)((6-(dimethylamino)-9-(4-(((2,5-dioxopyrrolidin-1-yl)oxy)carbonyl)-2-methylphenyl)-3H-xanthenylidene)(methyl)ammonio)propane-1-sulfonate: Under N₂, a scintillation vial was charged with (E)-3-((9-(4-carboxy-2-methylphenyl)-6-(dimethylamino)-3H-xanthen-3-ylidene)(methyl) ammonio)propane-1-sulfonate (75 mg, 148 µmol, 1.0 equiv.) and TSTU (67 mg, 221 µmol, 1.5 equiv.). The vial was purged under N₂ for an additional 15 min before adding 1 mL anhydrous DMF, followed by DiPEA (77 µL, 442 µmol, 3.0 equiv.). The reaction mixture was covered from light and stirred at rt for 1 h. The crude product was purified by reverse phase prep-HPLC (10-90% MeCN/H₂O gradient with 0.1% TFA). The title compound (43 mg, 48%) was obtained as a dark red solid. HRMS(ESI) [M+H]⁺ m/z found 606.1907, calcd for C₃₁H₃₂N₃O₈S 606.1905.

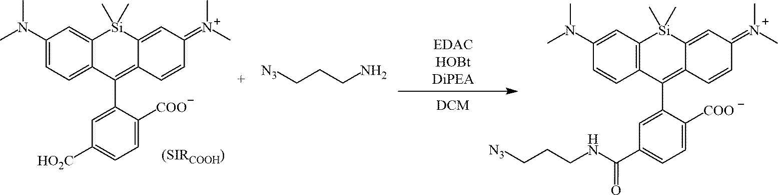

4-(azidopropyl)carbamoyl)-2-(7-(dimethylamino)-3-(dimethyliminio)-5,5-dimethyl-3,5-dihydrodibenzo[b,e]silin-10-yl)benzoate: Under N₂, 4-carboxy-2-(7-(dimethylamino)-3-(dimethyliminio)-5,5-dimethyl-3,5-dihydrodibenzo[b,e]silin-10-yl)benzoate (200 mg, 423 µmol, 1.0 equiv.) was dissolved in anhydrous DCM (20 mL). To the reaction flask, was added DiPEA (442 µL, 2.54 mmol, 6.0 equiv.), HOBt (71 mg, 508 µmol, 1.2 equiv.), 3-azidopropan-1-amine (85 mg, 846 µmol, 2.0 equiv.), and EDAC (98 mg, 502 µmol, 1.2 equiv.) in a sequential order. The reaction mixture was covered from light and stirred at rt overnight. The solvent was removed under reduced pressure, and the residue was purified by flash column chromatography in silica gel with DCM/EtOAc/MeOH (80/18/2) as eluent to give compound 59 (173 mg, 74%) as a light green solid. HRMS(ESI) [M+H]⁺ m/z found 555.2673, calcd for C₃₀H₃₅N₆O₃Si 555.2534.

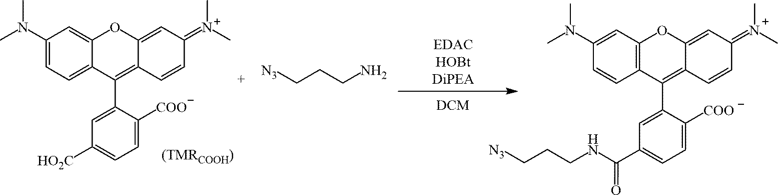

4-(azidopropyl)carbamoyl)-2-(6-(dimethylamino)-3-(dimethyliminio)-3H-xanthen-9-yl)benzoate: Under N₂, 4-carboxy-2-(6-(dimethylamino)-3-(dimethyliminio)-3H-xanthen-9-yl)benzoate (200 mg, 465 µmol, 1.0 equiv.) was dissolved in anhydrous DCM (20 mL). To the reaction flask, was added DiPEA (486 µL, 2.79 mmol, 6.0 equiv.), HOBt (78 mg, 558 µmol, 1.2 equiv.), 3-azidopropan-1-amine (93 mg, 929 µmol, 2.0 equiv.), and EDAC (107 mg, 558 µmol, 1.2 equiv.) in a sequential order. The reaction mixture was covered from light and stirred at rt overnight. The solvent was removed under reduced pressure, and the residue was purified by flash column chromatography in silica gel with DCM/MeOH/NH₄OH (95/5/2) as eluent to give the title compound (134 mg, 56%) as a dark red solid. HRMS(ESI) [M+H]⁺ m/z found 513.2269, calcd for C₂₈H₂₉N₆O₄ 513.2245.

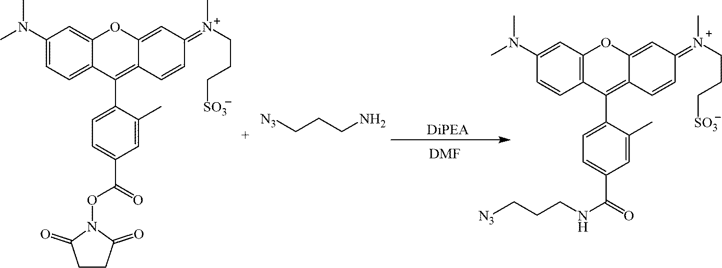

(E)((9-(4-((3-azidopropyl)carbamoyl)-2-methylphenyl)-6-(dimethylamino)-3H-xanthenylidene)(methyl)ammonio)propane-1-sulfonate: Under N₂, a scintillation vial was charged with (E)-3-((6-(dimethylamino)-9-(4-(((2,5-dioxopyrrolidin-1-yl)oxy)carbonyl)-2-methylphenyl) -3H-xanthen-3-ylidene)(methyl)ammonio)propane-1-sulfonate (20 mg, 33 µmol, 1.0 equiv.) and 3-azido-1-propanamine (6.6 mg, 66 µmol, 2.0 equiv.). The vial was purged under N₂ for an additional 15 min before adding 0.5 mL anhydrous DMF, followed by DiPEA (17 µL, 99 µmol, 3.0 equiv.). The reaction mixture was covered from light and stirred at rt for 12 h. The crude product was purified by reverse phase prep-HPLC (10-90% MeCN/H₂O gradient with 0.1% TFA). The title compound (17 mg, 87%) was obtained as a dark red solid. HRMS(ESI) [M+H]⁺ m/z found 591.2370, calcd for C₃₀H₃₅N₆O₅S 591.2384.

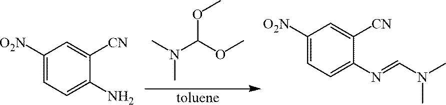

(E)-N′-cyano-4-nitrophenyl)-N,N-dimethylformimidamide: In a 250 mL round bottom flask, 2-amino-5-nitrobenzonitrile (5.0 g, 30.7 mmol, 1.0 equiv.) and N,N-Dimethylformamide dimethyl acetal (6.11 mL, 46.0 mmol, 1.5 equiv.) were suspended toluene (50 mL) before the reaction was placed on an oil bath, heated to ca. 110° C. and allowed to reflux for 3 hours. The reaction mixture was then removed from the oil bath and stirred under ambient conditions overnight. Upon cooling, the precipitate was collected via vacuum filtration, washed with Et₂0 (20 mL), air dried in the funnel overnight. The title compound (5.71 g, 85%) was obtained as a bright yellow solid. HRMS(ESI) [M+H]⁺ m/z found 219.0904, calcd for C₁₀H₁₁N₄O₂ 219.0877.

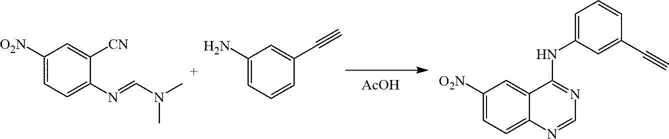

N-ethynylphenyl)-6-nitroquinazolin-4-amine: In a 250 mL round bottom flask, (E)-N′-(2-cyano-4-nitrophenyl)-N,N-dimethylformimidamide (4.0 g, 18.3 mmol, 1.0 equiv.) and 3-ethynylaniline (2.32 mL, 20.2 mmol, 1.1 equiv.) were suspended acetic acid (40 mL) before the reaction was placed on an oil bath, heated to ca. 120° C. and allowed to reflux for 2 hours. Upon cooling to rt, the formed precipitate was collected via vacuum filtration, washed with Et₂O (20 mL), air dried in the funnel overnight. The title compound (4.1 g, 92%) was obtained as a solid. HRMS(ESI) [M+H]⁺ m/z found 291.0903, calcd for C₁₆H₁₁N₄O₂ 291.0877.

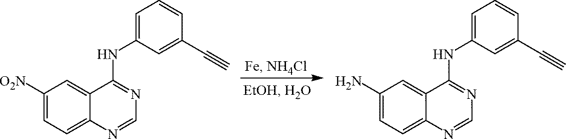

N⁴-ethynylphenyl)quinazoline-4,6-diamine: In a 100 mL round bottom flask, N-(3-ethynylphenyl)-6-nitroquinazolin-4-amine (2.0 g, 6.89 mmol, 1.0 equiv.), iron powder (2.12 g, 37.9 mmol, 5.5 equiv.), and NH₄Cl (277 mg, 5.17 mmol, 0.75 equiv.) were suspended in EtOH (90 mL) and water (10 mL) mixture before the reaction was placed on an oil bath, heated to ca. 90° C. and allowed to reflux for 3 hours. Upon cooling to rt, the solid was removed via vacuum filtration. The filtrate was washed with MeOH (10 mL), concentrated in vacuo. The residue was resuspended in water (50 mL), extracted with DCM (3 × 50 mL). The organic layers were combined, dried over anhydrous Na₂SO₄, filtered, and concentrated in vacuo. The title compound (1.69 g, 94%) was obtained as a solid. HRMS(ESI) [M+H]⁺ m/z found 261.1157, calcd for C₁₆H₁₃N₄ 261.1135.

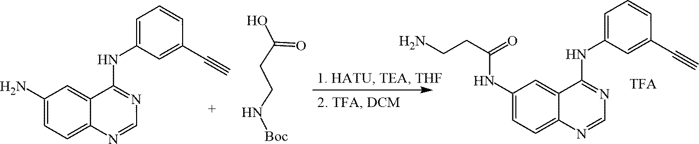

3-amino-N-(4-((3-ethynylphenyl)amino)quinazolin-6-yl)propenamide: Under N₂, N⁴-(3-ethynylphenyl)quinazoline-4,6-diamine (1.0 g, 3.84 mmol, 1.0 equiv.) and 3-((tert-butoxycarbonyl)amino)propanoic acid (800 mg, 4.23 mmol, 1.1 equiv.) were suspended in anhydrous THF (30 mL) before placed in an ice bath. At 0° C., HATU (2.19 g, 5.76 mmol, 1.5 equiv.) and TEA (643 µL, 4.61 mmol, 1.2 equiv.) were added to the reaction flask. The mixture was then removed from the cold bath and stirred under ambient conditions overnight. Upon reaction completion, the mixture was diluted with EtOAc (50 mL). The organic layer was then washed with water (50 mL), brine (50 mL), dried over anhydrous Na₂SO₄, filtered, and concentrated in vacuo. The residue was then redissolved in DCM (20 mL). The reaction solution was chilled in an ice bath for ca. 20 minutes before TFA (3.45 mL) was slowly added. The reaction mixture was stirred in the ice bath for 2.5 hours before concentrated in vacuo. The crude product was purified by reverse phase prep-HPLC (10-90% MeCN/H₂O gradient with 0.1% TFA). The title compound (493 mg, 39%) was obtained as an off white solid. HRMS(ESI) [M+H]⁺ m/z found 332.1531, calcd for C₁₉H₁₈N₅O 332.1506.

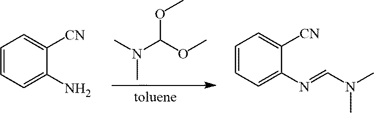

(E)-N′-cyanophenyl)-N,N-dimethylformimidamide: In a 100 mL round bottom flask, 2-aminobenzonitrile (2.5 g, 21.2 mmol, 1.0 equiv.) and N N-Dimethylformamide dimethyl acetal (4.22 mL, 31.7 mmol, 1.5 equiv.) were suspended toluene (25 mL) before the reaction was placed on an oil bath, heated to ca. 110° C. and allowed to reflux for 3 hours. The reaction mixture was then removed from the oil bath and stirred under ambient conditions overnight. Upon cooling, the precipitate was collected via vacuum filtration, washed with Et₂0 (10 mL), air dried in the funnel overnight. The title compound (2.85 g, 78%) was obtained as a solid. HRMS(ESI) [M+H]⁺ m/z found 174.1170, calcd for C_{1O}H₁₂N₃ 174.1026.

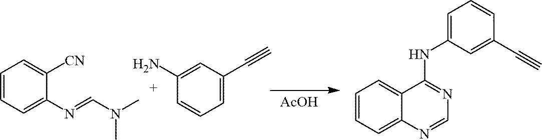

N-ethynylphenyl)quinazolin-4-amine: In a 250 mL round bottom flask, (E)-N′-(2-cyanophenyl)-N,N-dimethylformimidamide (2.0 g, 11.6 mmol, 1.0 equiv.) and 3-ethynylaniline (1.59 mL, 13.9 mmol, 1.2 equiv.) were suspended acetic acid (20 mL) before the reaction was placed on an oil bath, heated to ca. 120° C. and allowed to reflux for 2 hours. Upon cooling to rt, the formed precipitate was collected via vacuum filtration, washed with Et₂0 (10 mL), air dried in the funnel overnight. The title compound (2.22 g, 78%) was obtained as a solid. HRMS(ESI) [M+H]⁺ m/z found 246.1196, calcd for C₁₆H₁₂N₃ 246.1026.

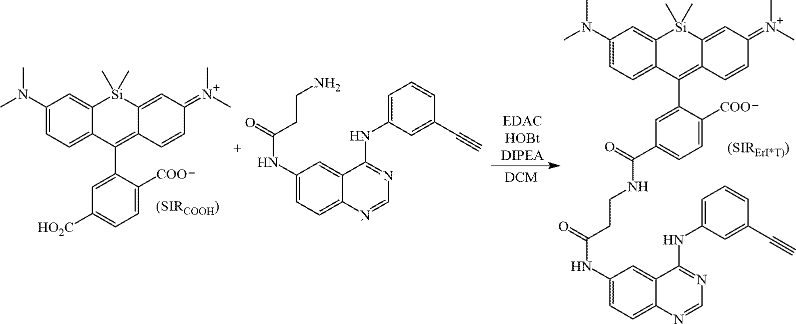

2-(dimethylamino)-3-(dimethyliminio)-5,5-dimethyl-3,5-dihydrodibenzo[b,e]silin-10-yl)-4-((3-((4-((3-ethynylphenyl)amino)quinazolin-6-yl)amino)-3-oxopropyl)carbamoyl)benzoate (SiR_{Erl(T)}): Under N₂, 4-carboxy-2-(7-(dimethylamino)-3-(dimethyliminio)-5,5-dimethyl-3,5-dihydrodibenzo[b,e]silin-10-yl)benzoate (SIR_{cooH})(20 mg, 42.3 µmol, 1.0 equiv.) was dissolved in anhydrous DCM (2 mL) in a scintillation vial. To the reaction vial, was added DiPEA (74 µL, 423 µmol, 10.0 equiv.), HOBt (18 mg, 127 µmol, 3.0 equiv.), EDAC (25 mg, 127 µmol, 3.0 equiv.), and then 3-amino-N-(4-((3-ethynylphenyl)amino)quinazolin-6-yl)propanamide (20 mg, 46.6 µmol, 1.1 equiv.) in a sequential order, approximately 5 minutes apart. The reaction mixture was covered from light and stirred at rt overnight. The solvent was removed under reduced pressure, and the residue was purified by reverse phase prep-HPLC (10-90% MeCN/H20 gradient with 0.1% TFA). The title compound (21 mg, 63%) was obtained as a blue solid. HRMS(ESI) [M+H]⁺ m/z found 786.3240, calcd for C₄₆H₄₄N₇O₄Si 786.3219.

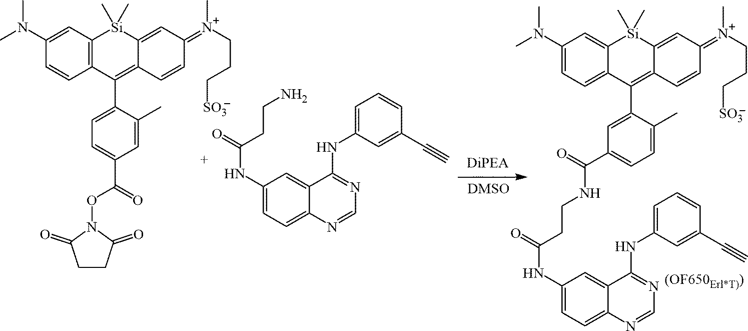

(E)((7-(dimethylamino)-10-(4-((3-((4-((3-ethynylphenyl)amino)quinazolin-6-yl)amino)oxopropyl)carbamoyl)-2-methylphenyl)-5,5-dimethyldibenzo[b,e]silin-3(5H)-ylidene)(methyl)ammonio)propane-1-sulfonate (OF650_{Erl(T)}): Under N₂, a 1 dram vial was charged with (E)-3-((7-(dimethylamino)-10-(4-(((2,5-dioxopyrrolidin-1-yl)oxy)carbonyl)-2-methylphenyl)-5,5-dimethyldibenzo[b,e]silin-3(5H)-ylidene)(methyl)ammonio)propane-1-sulfonate (20 mg, 31 µmol, 1.0 equiv.) and 3-amino-N-(4-((3-ethynylphenyl)amino)quinazolin-6-yl)propanamide (26 mg, 62 µmol, 2.0 equiv.). The vial was purged under N₂ for an additional 15 min before adding 0.5 mL anhydrous DMSO, followed by DiPEA (27 µL, 154 µmol, 5.0 equiv.). The reaction mixture was covered from light and stirred at rt for 4 h. The crude product was purified by reverse phase prep-HPLC (10-90% MeCN/H₂0 gradient with 0.1% TFA). The title compound (17 mg, 65%) was obtained as a dark blue solid. HRMS(ESI) [M+H]⁺ m/z found 864.3397, calcd for C₄₈H₅₀N₇O₅SSi 864.3358.

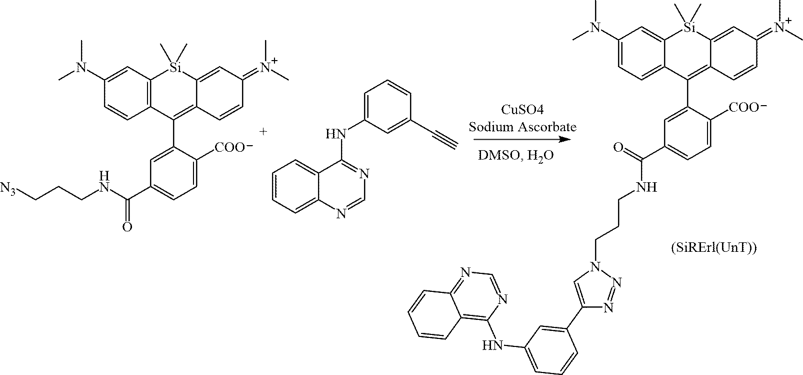

2-(dimethylamino)-3-(dimethyliminio)-5,5-dimethyl-3,5-dihydrodibenzo[b,e]silin-10-yl)-4-((3-(4-(3-(quinazolin-4-ylamino)phenyl)-1H-1,2,3-triazol-1-yl)propyl)carbamoyl)benzoate (SiR_{Erl(UnT)}): Under N2, a 1 dram vial was charged with compounds 4-((3-azidopropyl) carbamoyl)-2-(7-(dimethylamino)-3-(dimethyliminio)-5,5-dimethyl-3,5-dihydro dibenzo[b,e] silin-10-yl)benzoate (15 mg, 27 µmol, 1.0 equiv.) and N-(3-ethynylphenyl)quinazolin-4-amine (10 mg, 40.6 µmol, 1.5 equiv.), and degassed DMSO/H20 (1/1, v/v, 1 mL). To which, freshly prepared sodium 100 mM ascorbate solution (270 µL, 1.0 equiv.) and 100 mM CuSO₄ solution (54 µL, 0.2 equiv.) were added. The reaction mixture was covered from light and stirred at rt overnight. The crude product was purified by reverse phase prep-HPLC (10-90% MeCN/H₂O gradient with 0.1% TFA). The title compound (12 mg, 55%) was obtained as a light blue solid. HRMS(ESI) [M+H]⁺ m/z found 800.3433, calcd for C₄₆H₄₆N₉O₃Si 800.3487.

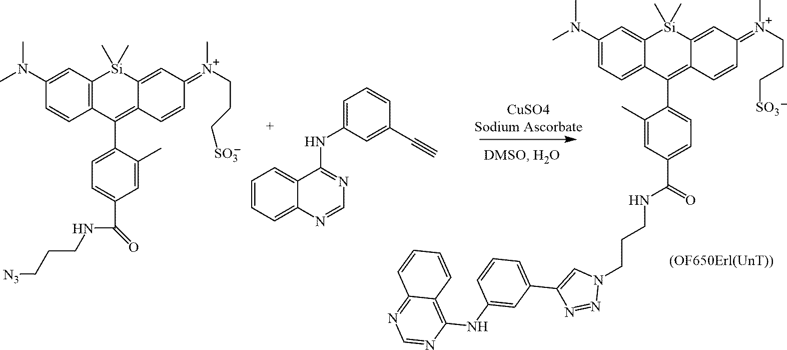

(E)((7-(dimethylamino)-5,5-dimethyl-10-(2-methyl-4-((3-(4-(3-(quinazolin-4-ylamino)phenyl)-1H-1,2,3-triazol-1-yl)propyl)carbamoyl)phenyl)dibenzo[b,e]silin-3(5H)-ylidene)(methyl)ammonio)propane-1-sulfonate (OF650_{Erl(UnT)}): Under N2, a 1 dram vial was charged with (E)-3-((10-(4-((3-azidopropyl)carbamoyl)-2-methylphenyl)-7-(dimethylamino)-5,5-dimethyldibenzo[b,e]silin-3(5H)-ylidene)(methyl)ammonio)propane-1-sulfonate (10 mg, 15.8 µmol, 1.0 equiv.) and N-(3-ethynylphenyl)quinazolin-4-amine (6 mg, 23.7 µmol, 1.5 equiv.), and degassed DMSO/H20 (1/1, v/v, 1 mL). To which, freshly prepared sodium 100 mM ascorbate solution (237 µL, 1.5 equiv.) and 100 mM CuSO₄ solution (79 µL, 0.5 equiv.) were added. The reaction mixture was covered from light and stirred at rt overnight. The crude product was purified by reverse phase prep-HPLC (10-90% MeCN/H₂O gradient with 0.1% TFA). The title compound (9.8 mg, 71%) was obtained as a dark blue solid. HRMS(ESI) [M+H]⁺ m/z found 878.3600, calcd for C₄₈H₅₂N₉O₄SSi 878.3627.

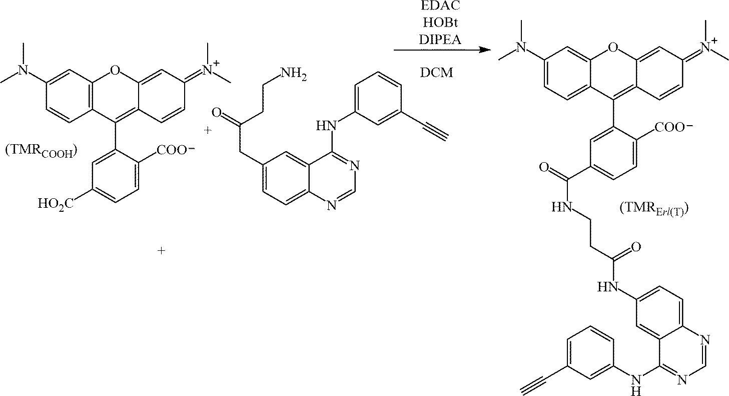

2-(dimethylamino)-3-(dimethyliminio)-3H-xanthen-9-yl)-4-((3-((4-((3-ethynylphenyl)amino)quinazolin-6-yl)amino)-3-oxopropyl)carbamoyl)benzoate (TMR_{Erl(T)}): Under N₂, 4-carboxy-2-(6-(dimethylamino)-3-(dimethyliminio)-3H-xanthen-9-yl)benzoate (20 mg, 46.5 µmol, 1.0 equiv.) was dissolved in anhydrous DCM (2 mL) in a scintillation vial. To the reaction vial, was added DiPEA (81 µL, 465 µmol, 10.0 equiv.), EDAC (27 mg, 139 µmol, 3.0 equiv.), HOBt (19 mg, 139 µmol, 3.0 equiv.), and then 3-amino-N-(4-((3-ethynylphenyl)amino)quinazolin-6-yl)propanamide (22 mg, 51.1 µmol, 1.1 equiv.) in a sequential order, approximately 5 minutes apart. The reaction mixture was covered from light and stirred at rt overnight. The solvent was removed under reduced pressure, and the residue was purified by reverse phase prep-HPLC (10-90% MeCN/H₂O gradient with 0.1% TFA). The title compound (17.7 mg, 51%) was obtained as a red solid. HRMS(ESI) [M+H]⁺ m/z found 744.2940, calcd for C₄₄H₃₈N₇₀₅ 744.2929.

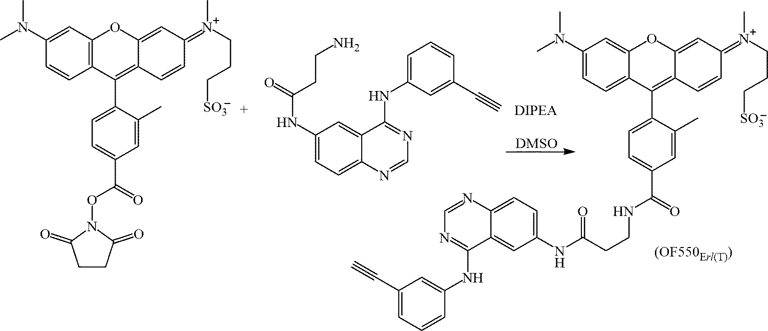

(E)((6-(dimethylamino)-9-(4-((3-((4-((3-ethynylphenyl)amino)quinazolin-6-yl)amino)oxopropyl)carbamoyl)-2-methylphenyl)-3H-xanthenylidene)(methyl)ammonio)propane-1-sulfonate (OF550_{Erl(T)}): Under N₂, a 1 dram vial was charged with (E)-3-((6-(dimethylamino)-9-(4-(((2,5-dioxopyrrolidin-1-yl)oxy)carbonyl)-2-methylphenyl)-3H-xanthen-3-ylidene)(methyl)ammonio)propane-1-sulfonate (20 mg, 33 µmol, 1.0 equiv.) and 3-amino-N-(4-((3-ethynylphenyl)amino)quinazolin-6-yl)propanamide (28 mg, 66 µmol, 2.0 equiv.). The vial was purged under N₂ for an additional 15 min before adding 0.5 mL anhydrous DMSO, followed by DiPEA (23 µL, 165 µmol, 5.0 equiv.). The reaction mixture was covered from light and stirred at rt for 4 h. The crude product was purified by reverse phase prep-HPLC (10-90% MeCN/H₂O gradient with 0.1% TFA). The title compound (19 mg, 71%) was obtained as a dark blue solid. HRMS(ESI) [M+H]⁺ m/z found 822.3053, calcd for C₄₆H₄₄N₇O₆S 822.3068.

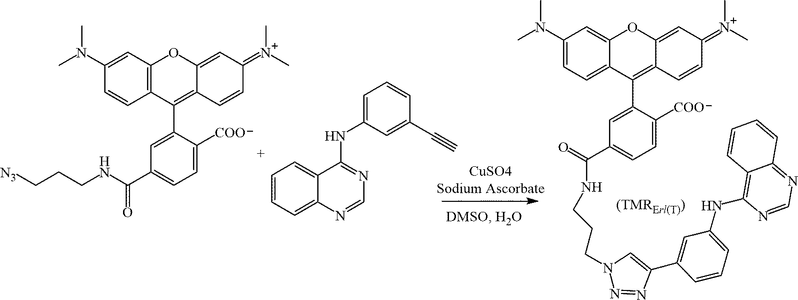

2-(dimethylamino)-3-(dimethyliminio)-3H-xanthen-9-yl)-4-((3-(4-(3-(quinazolin-4-ylamino)phenyl)-1H-1,2,3-triazol-1-yl)propyl)carbamoyl)benzoate (TMR_{Erl(UnT)}): Under N₂, a 1 dram vial was charged with 4-((3-azidopropyl)carbamoyl)-2-(6-(dimethylamino)-3-(dimethyliminio)-3H-xanthen-9-yl)benzoate (15 mg, 29.3 µmol, 1.0 equiv.) and N-(3-ethynylphenyl)quinazolin-4-amine (11 mg, 43.9 µmol, 1.5 equiv.), and degassed DMSO/H20 (1/1, v/v, 1 mL). To which, freshly prepared sodium 100 mM ascorbate solution (293 µL, 1.0 equiv.) and 100 mM CuSO₄ solution (59 µL, 0.2 equiv.) were added. The reaction mixture was covered from light and stirred at rt overnight. The crude product was purified by reverse phase prep-HPLC (10-90% MeCN/H₂0 gradient with 0.1% TFA). The title compound (11 mg, 51%) was obtained as a red solid. HRMS(ESI) [M+H]⁺ m/z found 758.3186, calcd for C₄₄H₄₀N₉O₄ 758.3198.

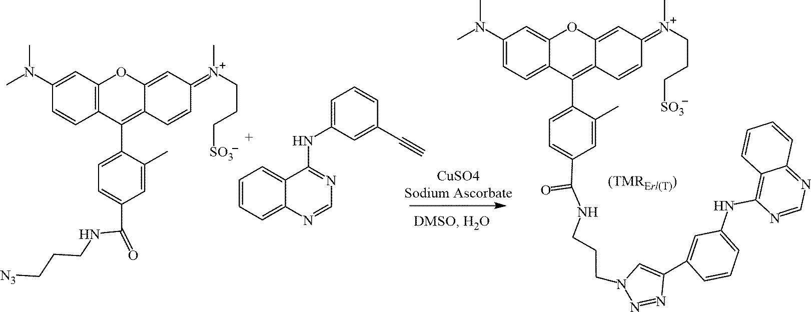

(E)((6-(dimethylamino)-9-(2-methyl-4-((3-(4-(3-(quinazolin-4-ylamino)phenyl)-1H-1,2,3-triazol-1-yl)propyl)carbamoyl)phenyl)-3H-xanthenylidene)(methyl)ammonio)propane-1-sulfonate (OF550_{Erl(UnT)}): Under N2, a 1 dram vial was charged with compounds (E)-3-((9-(4-((3-azidopropyl)carbamoyl)-2-methylphenyl)-6-(dimethylamino)-3H-xanthen-3-ylidene)(methyl) ammonio)propane-1-sulfonate (15 mg, 25.4 µmol, 1.0 equiv.) and N-(3-ethynylphenyl)quinazolin-4-amine (9 mg, 38.1 µmol, 1.5 equiv.), and degassed DMSO/H20 (1/1, v/v, 1 mL). To which, freshly prepared sodium 100 mM ascorbate solution (305 µL, 1.2 equiv.) and 100 mM CuSO₄ solution (127 µL, 0.5 equiv.) were added. The reaction mixture was covered from light and stirred at rt overnight. The crude product was purified by reverse phase prep-HPLC (10-90% MeCN/H₂O gradient with 0.1% TFA). The title compound (13 mg, 63%) was obtained as a red solid. HRMS(ESI) [M+H]⁺ m/z found 836.3421, calcd for C₄₆H₄₆N₉O₅S 836.3337.

### REFERENCES

De Niz et al., Progress in imaging methods: insights gained into Plasmodium biology. Nature Reviews Microbiology 2017, 15 (1), 37-54.

2. Nicovich et al., Turning single-molecule localization microscopy into a quantitative bioanalytical tool. Nature protocols 2017, 12 (3), 453-460.

3. Sahl et al., Fluorescence nanoscopy in cell biology. Nature Reviews Molecular Cell Biology 2017, 18 (11), 685-701.

4. Vicidomini et al., STED super-resolved microscopy. Nature Methods 2018, 15 (3), 173-182.

5. Xiao, J.; Dufrêne, Y. F., Optical and force nanoscopy in microbiology. Nature microbiology 2016, 1 (11), 16186.

6. Jradi et al., Chemistry of Photosensitive Fluorophores for Single-Molecule Localization Microscopy. ACS Chemical Biology 2019.

7. Grimm et al., A general method to improve fluorophores for live-cell and single-molecule microscopy. Nat Methods 2015, 12 (3), 244-50, 3 p following 250.

8. Grimm et al., Bright photoactivatable fluorophores for single-molecule imaging. Nat Methods 2016, 13 (12), 985-988.

9. Grimm et al., A general method to fine-tune fluorophores for live-cell and in vivo imaging. Nat Methods 2017, 14 (10), 987-994.

10. Choi et al., Targeted zwitterionic near-infrared fluorophores for improved optical imaging. Nat Biotechnol 2013, 31 (2), 148-53.

11. Hyun et al., Structure-inherent targeting of near-infrared fluorophores for parathyroid and thyroid gland imaging. Nat Med 2015, 21 (2), 192-7.

12. Day et al., The fluorescent protein palette: tools for cellular imaging. Chemical Society reviews 2009, 38 (10), 2887-2921.

13. Hoffman, R. M., The multiple uses of fluorescent proteins to visualize cancer in vivo. Nature Reviews Cancer 2005, 5 (10), 796-806.

14. Rodriguez et al.,The Growing and Glowing Toolbox of Fluorescent and Photoactive Proteins. Trends in Biochemical Sciences 2017, 42 (2), 111-129.

15. Oliinyk et al., Smallest near-infrared fluorescent protein evolved from cyanobacteriochrome as versatile tag for spectral multiplexing. Nature Communications 2019, 10 (1), 279.

16. Heilemann et al.,Super-Resolution Imaging with Small Organic Fluorophores. Angewandte Chemie International Edition 2009, 48 (37), 6903-6908.

17. Dempsey et al., Evaluation of fluorophores for optimal performance in localization-based super-resolution imaging. Nature Methods 2011, 8 (12), 1027-1036.

18. Fölling et al., Fluorescence nanoscopy by ground-state depletion and single-molecule return. Nature Methods 2008, 5 (11), 943-945.

19. Maiti, et al., Orthogonal Cu— and Pd-based catalyst systems for the O— and N-arylation of aminophenols. J Am Chem Soc 2009, 131 (47), 17423-9.

20. Hanaoka et al.,Synthesis of unsymmetrical Si-rhodamine fluorophores and application to a far-red to near-infrared fluorescence probe for hypoxia. Chem Commun (Camb) 2018, 54 (50), 6939-6942.

Sung, J. et al. A New Infrared Probe Targeting Mitochondria via Regulation of Molecular Hydrophobicity. Bioconjugate Chemistry 30, 210-217 (2019).

Chu, Y. et al. Development of Theragnostic Tool Using NIR Fluorescence Probe Targeting Mitochondria in Glioma Cells. Bioconjugate Chemistry 30, 1642-1648 (2019).

Kim, E., Yang, K.S., Giedt, R.J. & Weissleder, R. Red Si-rhodamine drug conjugates enable imaging in GFP cells. Chemical Communications 50, 4504-4507 (2014).

Numasawa, K. et al. A Fluorescent Probe for Rapid, High-Contrast Visualization of Folate-Receptor-Expressing Tumors In Vivo. Angewandte Chemie International Edition 59, 6015-6020 (2020).

Johnson, I. Review: Fluorescent probes for living cells. The Histochemical Journal 30, 123-140 (1998).

Rurack et al., Fluorescence Quantum Yields of a Series of Red and Near-Infrared Dyes Emitting at 600-1000 nm. Analytical Chemistry 83, 1232-1242 (2011).

Koide et al., Evolution of Group 14 Rhodamines as Platforms for Near-Infrared Fluorescence Probes Utilizing Photoinduced Electron Transfer. ACS Chemical Biology 6, 600-608 (2011).

Bachman et al., Improved Xanthone Synthesis, Stepwise Chemical Redox Cycling. Organic Letters 21, 206-209 (2019).

Maiti et al.,Orthogonal Cu— and Pd-based catalyst systems for the O— and N-arylation of aminophenols. J Am Chem Soc 131, 17423-17429 (2009).

Lukinavičius et al. A near-infrared fluorophore for live-cell super-resolution microscopy of cellular proteins. Nature Chemistry 5, 132-139 (2013).

Kvach, M.V. et al. Practical Synthesis of Isomerically Pure 5- and 6-Carboxytetramethylrhodamines, Useful Dyes for DNA Probes. Bioconjugate Chemistry 20, 1673-1682 (2009).

### Detailed Descriptions of Figures

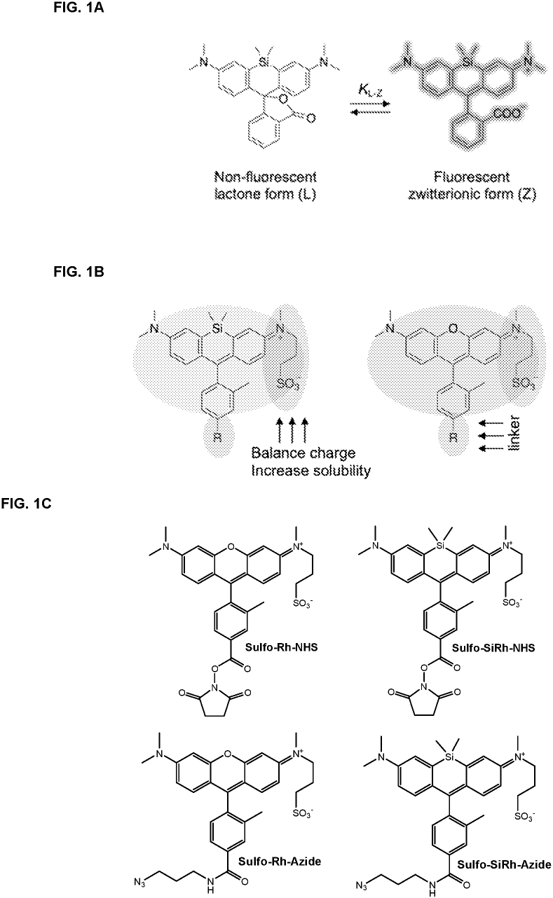

FIG. 1: Zwitterionic cell-permeant and water-soluble rhodamine dyes structural design. A. Conventional Silicon-substituted rhodamine (SiTMR) exhibits environmentally sensitive intramolecular cyclization properties when placed in a hydrophobic environment. This structural change results because the carboxylate anion attacks the central carbon forming a spirolactone, which is both colorless and non-fluorescent, not ideal for quantitative imaging applications. B. To eliminate this structural change we synthesized sulfonated versions of TMR and Si-TMR, termed Sulfo-Rh and Sulfo-SiRh where the carboxylic acid moiety that formed the spirolactone was removed to prevent the fluorescence on-off equilibrium; the resulting cationic charge is balanced by attaching one sulfonate group to the far end of the N-alkyl group. C. Synthetic sequences for the reactive versions of water-soluble and cell-permeable fluorophores, including the N-hydroxysuccinimide (NHS) ester and azido reactive versions have also been developed, providing utility for bioconjugation reactions (e.g., antibodies, small molecules, etc.).

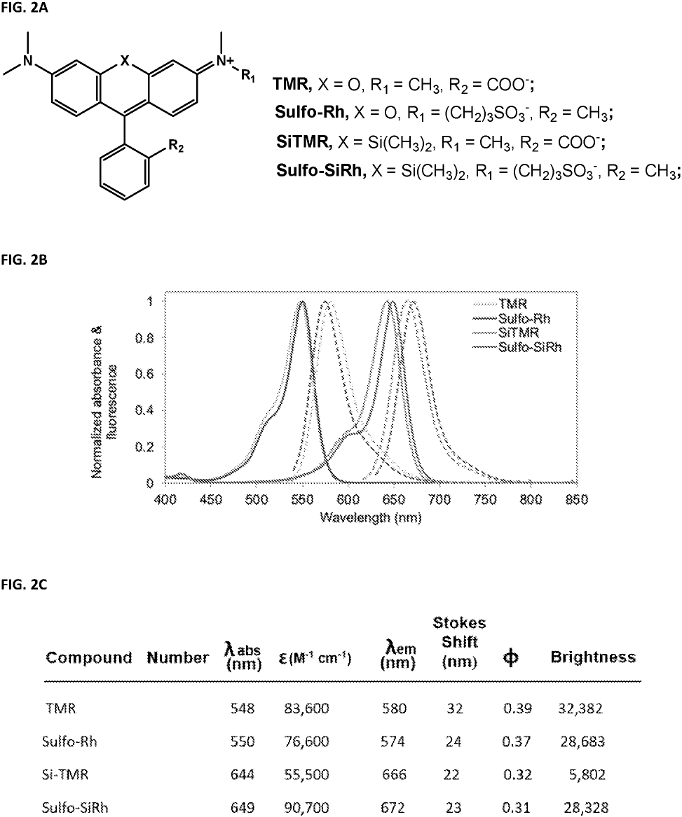

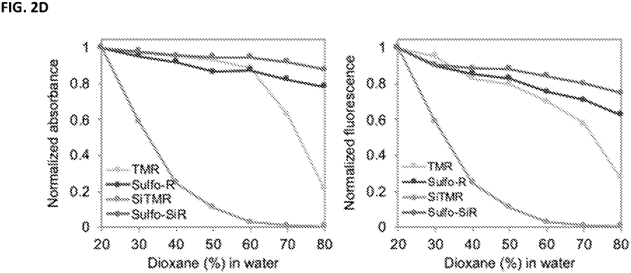

FIG. 2: Zwitterionic Cell-Permeant and Water-Soluble Rhodamine Shows Improved Fluorescence Stability Compared to Conventional rhodamines. A. general rhodamine fluorophore structures including the classic TMR, SiTMR, and the sulfonated versions. Conventional Si-TMR exhibits environmentally sensitive cyclization properties when placed in a hydrophobic environment. This structural change results because the carboxylate anion attacks the central carbon forming a spirolactone, which is both colorless and non-fluorescent. B. Normalized absorbance and fluorescence emission spectra of the four fluorophores in PBS buffer show the sulfonated versions exhibited minor maximum absorption and emission wavelength changes (< 10 nm) comparing to the base fluorophores (TMR and SiTMR) respectively. C. Tabulated spectral properties of all four fluorophores in PBS buffer show the Sulfo-SiRh exhibit brightness five time greater than the base fluorophore SiTMR. D. We demonstrated the stability of sulfonated rhodamine fluorophores vs. base TMR and SiTMR fluorophores by collecting absorption (left) and fluorescence (right) spectra with varied percentages of dioxane (20-80%) in DI water resulting the polarity of the screening solvent systems changing in a gradient manner. As expected, classic Si-TMR absorbance and fluorescence intensity both diminished rapidly with increasing percentages of dioxane. This change in both absorbance and fluorescence intensity was alleviated using our sulfonated versions.

The table below provides tabulated spectral properties of ORFluor-based dyes and probes in comparison to the TMR- and SiR-based dyes and labeled probes in aqueous media.

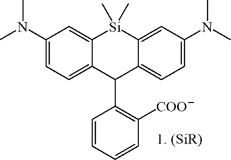

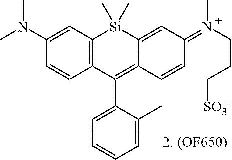

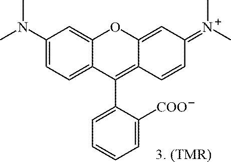

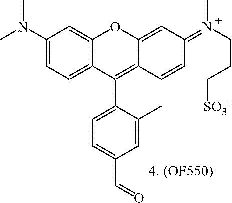

| Cmpd No. | Name | λabs (nm) | ε (M⁻¹ cm₋₁) | FWHM_{abs} (nm) | λₑₘ (nm) | Stokes Shift (nm) | FWHₑₘ (nm) | ϕ | Brightness |
|---|---|---|---|---|---|---|---|---|---|
| 1 | SiR | 642 | 19,200 | 39 | 662 | 20 | 41 | 0.35 | 7 |
| 2 | OF650 | 649 | 125,500 | 33 | 668 | 19 | 40 | 0.44 | 55 |
| 5 | SiR_{COOH} | 645 | 64,700 | 39 | 667 | 22 | 42 | 0.45 | 29 |
| 6 | OF650_{COOH} | 650 | 200,600 | 34 | 672 | 22 | 42 | 0.41 | 82 |
| 9 | SiR_{HALO} | 646 | 11,100 | 45 | 664 | 18 | 45 | 0.25 | 3 |
| 10 | OF650_{HALO} | 653 | 145,700 | 33 | 674 | 21 | 41 | 0.33 | 48 |
| 11 | OF650_{SNAP} | 653 | 82,000 | 35 | 671 | 18 | 40 | 0.24 | 20 |
| 15 | SiR_{Erl(T)} | 656 | 34,800 | 42 | 678 | 22 | 53 | 0.13 | 5 |
| 16 | OF650_{Erl(T)} | 656 | 43,400 | 38 | 673 | 17 | 41 | 0.16 | 7 |
| 17 | SiR_{Erl(UnT)} | 654 | 24,100 | 38 | 670 | 16 | 43 | 0.19 | 5 |
| 18 | OF650_{Erl(UnT)} | 656 | 100,500 | 33 | 674 | 18 | 42 | 0.35 | 35 |
| 3 | TMR | 549 | 60,900 | 38 | 574 | 25 | 42 | 0.54 | 33 |
| 4 | OF550 | 552 | 97,300 | 33 | 575 | 23 | 40 | 0.61 | 59 |
| 7 | TMR_{COOH} | 550 | 83,800 | 42 | 576 | 26 | 44 | 0.58 | 49 |
| 8 | OF550_{COOH} | 554 | 112,600 | 33 | 578 | 24 | 42 | 0.69 | 78 |
| 12 | TMR_{SNAP} | 553 | 27,700 | 58 | 577 | 24 | 45 | 0.41 | 11 |
| 13 | OF550_{SNAP} | 556 | 59,500 | 38 | 580 | 24 | 43 | 0.36 | 21 |
| 14 | OF550_{HALO} | 556 | 97,200 | 34 | 581 | 25 | 42 | 0.55 | 53 |
| 19 | TMR_{Erl(T)} | 563 | 33,100 | 39 | 587 | 24 | 42 | 0.62 | 21 |
| 20 | OF550_{Erl(T)} | 566 | 24,500 | 80 | 583 | 17 | 47 | 0.28 | 7 |
| 21 | TMR_{Erl(UnT)} | 559 | 18,700 | 44 | 585 | 26 | 43 | 0.37 | 7 |
| 22 | OF550_{Erl(UnT)} | 560 | 50,200 | 35 | 585 | 25 | 44 | 0.69 | 35 |

OF650 and OF550 have shown substantially improved stability against solvent polarity changes compared to the base fluorophores SiR and TMR, respectively. In addition to matched structural features, these newly synthesized fluorophores also exhibited matched photophysical properties, including net charge, molecular size, Stokes’ shift, FWHM, and brightness. These matched spectral properties and improved stability against live cell and tissue environmental polarity changes, taken together with their matched structural features, effectively demonstrate the added utility of these water-soluble and cell membrane-permeable fluorophores, OF650 and OF550, for quantitative live cell imaging applications.

What is claimed:

1. A compound of Formula (I): 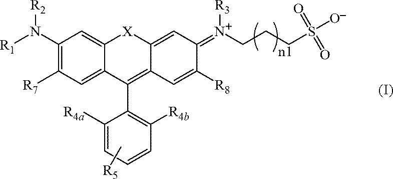
wherein:
X is selected from the group of O, —Si(CH₃)₂—, S, —N(CH₃)—, —C(CH₃)₂—, —P(OOH)—, —P(OPh)—, and —SO₂;
R₁ and R₂ are independently selected from the group of H and C₁-C₃ alkyl, with the proviso that at least one of R₁ and R₂ is not H;
or R₁ and R₂, together with the nitrogen atom to which they are bound, may form an azetidinyl ring, a pyrrolidinyl ring, or a piperidinyl ring, wherein the azetidinyl ring, pyrrolidinyl ring, or piperidinyl ring is substituted by 0, 1, 2, or 3 C₁-C₃ alkyl substituents;
R₃ is selected from the group of H and C₁-C₃ alkyl;
R₄ₐ and R_{4b} are each independently selected from the group of H and C₁-C₃ alkyl;
R₅ is bound to either the 3-position carbon atom or the 4-position carbon atom in the phenyl ring and is selected from the group of H, 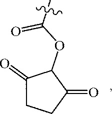 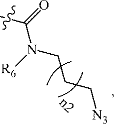 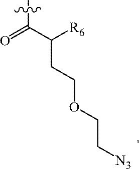 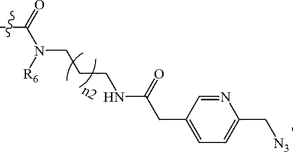 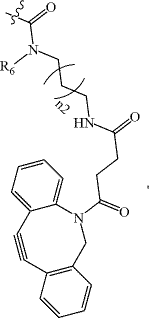 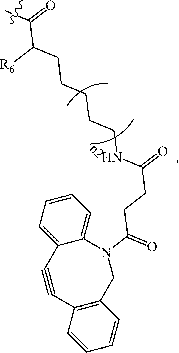 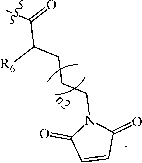 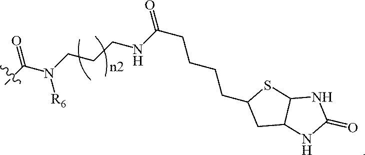 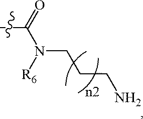 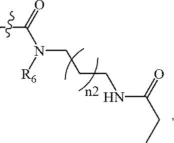 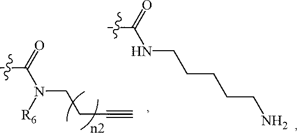 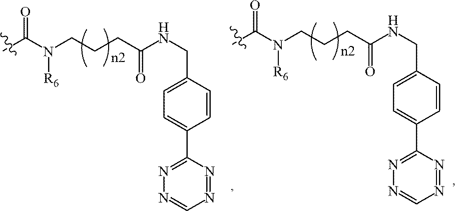 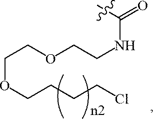 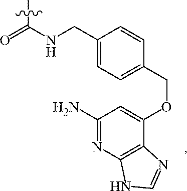 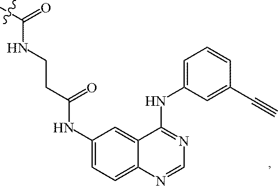 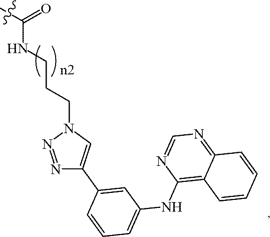 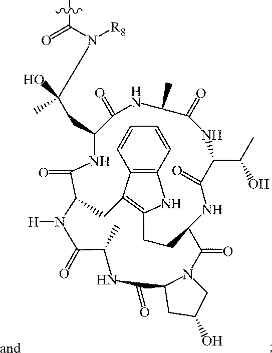
R₆ is selected from the group of H and C₁-C₃ alkyl;
n1 is an integer selected from the group of 0, 1, 2, and 3; and
n2 is an integer selected from the group of 0, 1, 2, 3, 4, 5, and 6;
R₇ is hydrogen;
R₈ is hydrogen;
or R₁ and R₇, together with the nitrogen atom to which R₁ is bound, form a 5-membered or 6-membered fused fully saturated or partially unsaturated nitrogen-containing heterocyclic ring, wherein the 6-membered fused fully saturated or partially unsaturated nitrogen-containing heterocyclic ring may have a further ring oxygen heteroatom to form a fused morpholinyl ring, and further wherein the 5-membered or 6-membered fused fully saturated or partially unsaturated nitrogen-containing heterocyclic ring is substituted by 0, 1, 2, 3, 4, or 5 C₁-C₃ alkyl substituents; or
or R₃ and R₈, together with the nitrogen atom to which R₁ is bound, form a 5-membered or 6-membered fused fully saturated or partially unsaturated nitrogen-containing heterocyclic ring, wherein the 6-membered fused fully saturated or partially unsaturated nitrogen-containing heterocyclic ring may have a further ring oxygen heteroatom to form a fused morpholinyl ring, and further wherein the 5-membered or 6-membered fused fully saturated or partially unsaturated nitrogen-containing heterocyclic ring is substituted by 0, 1, 2, 3, 4, or 5 C₁-C₃ alkyl substituents.

2. The compound of claim 1, wherein: wherein:
X is selected from the group of O, —Si(CH₃)₂—, S, —N(CH₃)—, —C(CH₃)₂—, —P(OOH)—, —P(OPh)—, and —SO₂;
R₁ and R₂ are independently selected from the group of H and C₁-C₃ alkyl, with the proviso that at least one of R₁ and R₂ is not H;
or R₁ and R₂, together with the nitrogen atom to which they are bound, may form an azetidinyl ring, a pyrrolidinyl ring, or a piperidinyl ring, wherein the azetidinyl ring, pyrrolidinyl ring, or piperidinyl ring is substituted by 0, 1, 2, or 3 C₁-C₃ alkyl substituents;
R₃ is selected from the group of H and C₁-C₃ alkyl;
R₄ₐ and R_{4b} are each independently selected from the group of H and C₁-C₃ alkyl;
R₅ is bound to either the 3-position carbon atom or the 4-position carbon atom in the phenyl ring and is selected from the group of H, 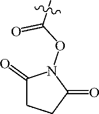 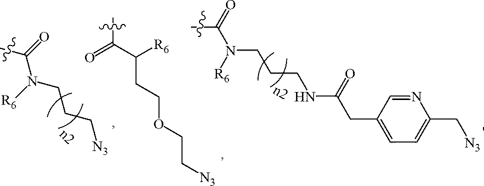 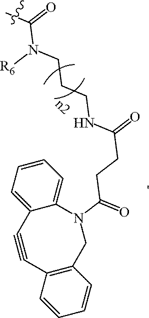 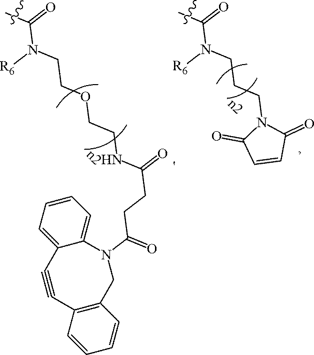 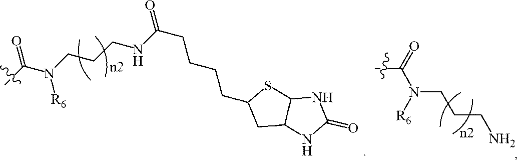 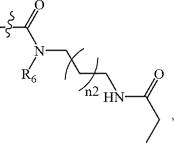 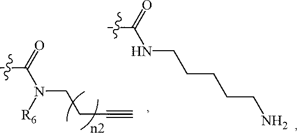 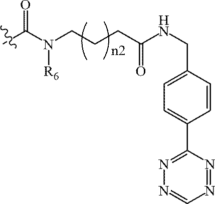 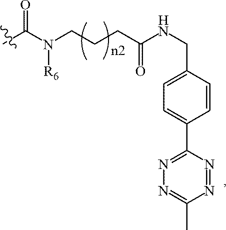 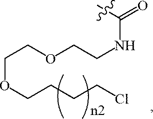 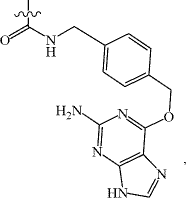 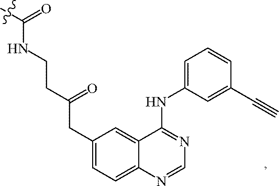 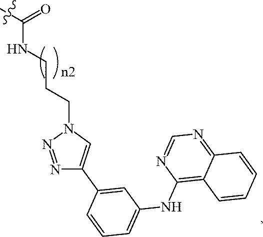 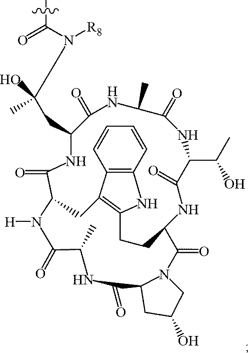
R₆ is selected from the group of H and C₁-C₃ alkyl;
n1 is an integer selected from the group of 0, 1, 2, and 3; and
n2 is an integer selected from the group of 0, 1, 2, 3, 4, 5, and 6; and
R₇ and R₈ are both hydrogen.

3. The compound of claim 1 of the formula: 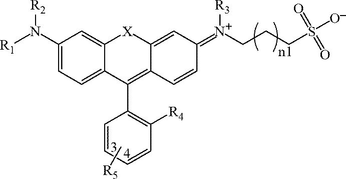
wherein:
X is selected from the group of O and —Si(CH₃)₂—;
R₁ and R₂ are independently selected from the group of H and C₁-C₃ alkyl, with the proviso that at least one of R₁ and R₂ is not H;
R₃ is selected from the group of H and C₁-C₃ alkyl;
R₄ is selected from the group of H, C₁-C₃ alkyl, —C(O)OH, and -C(O)O-C₁-C₃ alkyl;
R₄ is selected from the group of H and C₁-C₃ alkyl;
R₅ is bound to either the 3-position carbon atom or the 4-position carbon atom in the phenyl ring and is selected from the group of H, 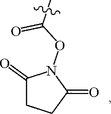 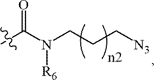 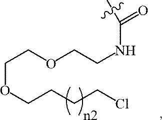 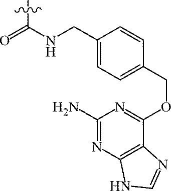 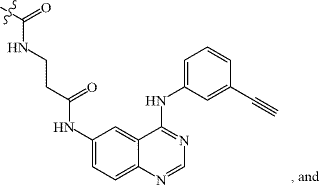 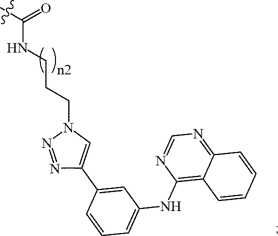
R₆ is selected from the group of H and C₁-C₃ alkyl; and
n1 and n2 are as defined in claim 1.

4. The compound of claim 3, wherein R₃ is C₁-C₃ alkyl.

5. The compound of claim 3, wherein R₃ is methyl.

6. The compound of claim 5 wherein R₄ is methyl.

7. The compound of claim 3, wherein R₅ is selected from the group of: 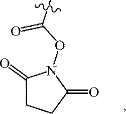 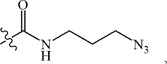 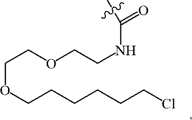 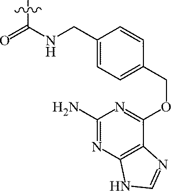 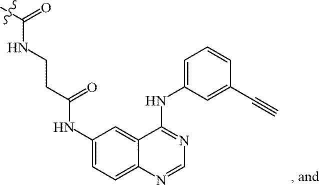 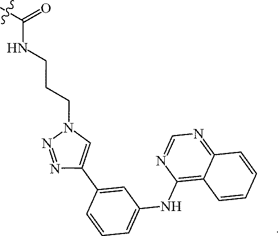
.

8. The compound of claim 1 selected from the group of: 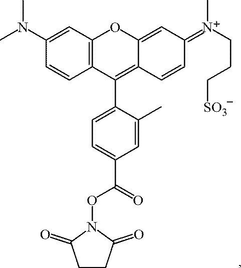 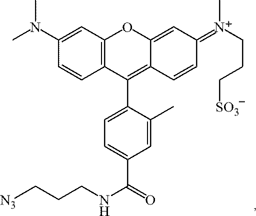 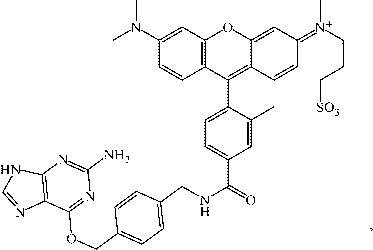 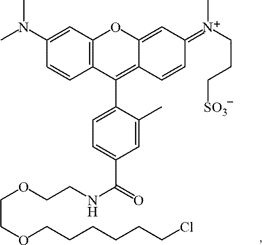 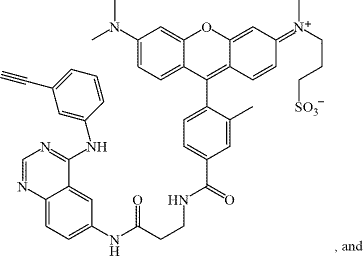 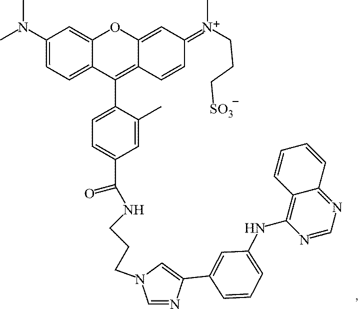
.

9. The compound of claim 1 selected from the group of: 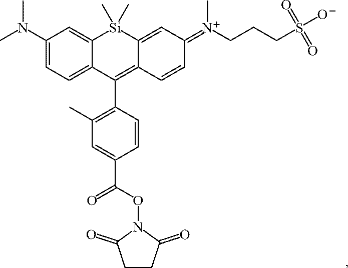 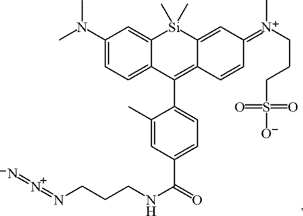 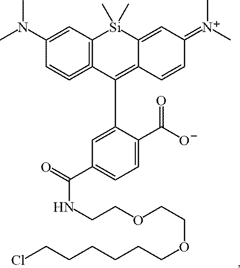 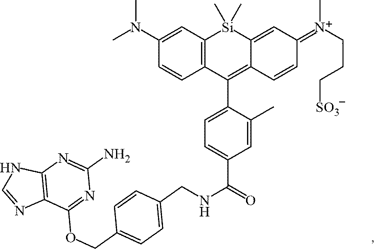 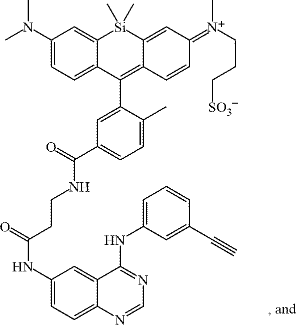 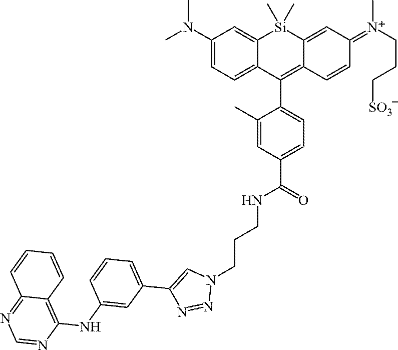
.

10. A pharmaceutical composition comprising an effective amount of a compound of claim 1 and a pharmaceutically acceptable carrier or excipient.

11. A method of labeling a biomolecule, cell, tissue, or organ of interest, the method comprising contacting the biomolecule, cell, tissue, or organ of interest with at least one fluorophore compound of claim 1.
